(19) United States
(12) Patent Application Publication
(10) Pub. No.: US 2019/0024060 A1
(43) Pub. Date: Jan. 24, 2019

(54) MICROORGANISMS OF THE GENUS CORYNEBACTERIUM HAVING L-ISOLEUCINE PRODUCING ABILITY AND METHODS FOR PRODUCING L-ISOLEUCINE USING THE SAME

(71) Applicant: CJ Cheiljedang Corporation, Seoul (KR)

(72) Inventors: Su Yon Kwon, Gyeonggi-do (KR); Jae Woo Jang, Gyeonggi-do (KR); Min Se Kim, Seoul (KR); Ju Jeong Kim, Gyeonggi-do (KR)

(21) Appl. No.: 15/768,497

(22) PCT Filed: Oct. 21, 2016
(86) PCT No.: PCT/KR2016/011923

§ 371 Date: Apr. 13, 2018

(30) Foreign Application Priority Data
Oct. 23, 2015 (KR) 10-2015-0148157

(51) Int. Cl. C12N 9/10; C12P 13/06; C12N 9/02; C12N 9/04; C12N 9/88

(52) U.S. Cl. CPC C12N 9/1029; C12P 13/06; C12Y 203/01182; C12N 9/0008; C12Y 402/01033; C12N 9/0006; C12Y 101/01085; C12N 9/88; C12Y 102/04001

(57) ABSTRACT

The present application relates to a microorganism of the genus Corynebacterium having L-isoleucine producing ability which comprises a protein having an activity of citramalate synthase, and a method for producing L-isoleucine using the same.

### TECHNICAL FIELD

The present disclosure relates to a microorganism of the genus Corynebacterium having L-isoleucine producing ability, and a method for producing L-isoleucine using the same.

### BACKGROUND ART

Branched-chain amino acids refer to the three amino acids L-valine, L-leucine, and L-isoleucine, and are industrially used as food additives, pharmaceutical agents, etc. In particular, L-isoleucine is metabolized in muscles to generate energy, and is involved in the generation of hemoglobin while functioning to relieve fatigue and promote growth. Accordingly, L-isoleucine has various uses as a fluid preparation, nutritional supplement, etc., and its use as a workout supplement is increasing.

L-isoleucine is biosynthesized from the precursors pyruvate and 2-ketobutyrate through three metabolic intermediates (Jinhwan Park et al., Appl. Microbial. Biotechnol. 85: 491-506, 2010).

However, in the biosynthesis of L-isoleucine, threonine dehydratase (ilvA gene), which produce 2-ketobutyrate from L-threonine, and then acetohydroxy acid synthase (ilvBN gene) are both subject to feedback inhibition by L-isoleucine.

Accordingly, it is known that regulation of enzymes involved in biosynthesis by release from feedback inhibition by L-isoleucine is an important element in preparing a strain producing a high yield of L-isoleucine (Jinhwan Park et al., Biotechnology Journal, 560-577, 2010). Further, branched-chain amino acids are generated through the same biosynthesis pathways as far as pyruvate, and accordingly, mass production of L-isoleucine at high yield requires smooth provision of L-threonine in the step prior to 2-ketobutyrate (Korean Patent No. 10-0823044).

### DISCLOSURE

### Technical Problem

The present inventors examined a novel biosynthesis pathway which does not use L-threonine as a precursor, and introduced a gene having an activity of citramalate synthase into the biosynthesis pathway of L-isoleucine. As a result, the inventors confirmed that the introduction improves the production of L-isoleucine, thereby completing the present disclosure.

### Technical Solution

An objective of the present disclosure is to provide a recombinant microorganism having an introduced novel biosynthesis pathway and having L-isoleucine producing ability.

Another objective of the present disclosure is to provide a method for producing L-isoleucine, comprising culturing there recombinant microorganism having an introduced novel biosynthesis pathway in a medium; and recovering L-isoleucine from the microorganism or medium.

### Advantageous Effect

The microorganism of the genus Corynebacterium having L-isoleucine producing ability of the present disclosure has an introduced activity of citramalate synthase, and can produce a high yield of L-isoleucine through a novel biosynthesis pathway where L-threonine is not used as a precursor.

### PREFERRED EMBODIMENT OF INVENTION

An aspect of the present disclosure to achieve the objects provides a microorganism of the genus Corynebacterium having L-isoleucine producing ability and comprising a protein having an activity of citramalate synthase.

The term “L-isoleucine” in the present disclosure refers to an essential amino acid, and structurally, to the L-amino acid of the formula HO₂CCH(NH₂)CH(CH₃)CH₂CH₃, corresponding to a branched-chain amino acid together with L-valine and L-leucine.

The phrase “having L-isoleucine producing ability” in the present disclosure indicates that when a related microorganism is cultured in a medium, it exhibits an ability to accumulate L-isoleucine in the medium or the microorganism itself. Such L-isoleucine producing ability may be inherited by a wild-type strain or provided with or enhanced by modifying a strain.

For example, for having L-isoleucine producing ability, the microorganism may adopt methods used for modification of a microorganism solely or in combination, such as acquisition of an auxotrophic, analogue resistant, or metabolism control mutant, or construction of a recombinant strain in which an activity of an enzyme involved in the L-isoleucine biosynthesis is enhanced.

Further, in the case of enhancing an activity of an enzyme involved in the L-isoleucine biosynthesis, the targeted gene involved in the L-isoleucine biosynthesis, specifically ilvG, ilvM, ilvE, ilvD, and ilvA genes, may be enhanced solely or in a combination of two or more genes. The ilvG, ilvM, ilvE, ilvD, and ilvA genes encode large and small subunits of isozyme II of acetohydroxy acid synthase, transaminase, dihydroxy acid dehydratase, and threonine deaminase, respectively.

The term “citramalate synthase (EC2.3.1.182)” in the present disclosure is a catalytic enzyme for transforming acetyl-CoA and pyruvate to citramalate and coenzyme A. This enzyme is found in Archaea such as Methanocaldococcus jannaschii (Howell et al., J Bacteriol. 181: 331-333, 1999), Leptospira interogans (Xu et al., J Bacteriol. 186: 5400-5409, 2004), and Geobacter sulfurreducens, and is involved in a threonine-independent pathway in the biosynthesis of isoleucine (Risso et al., J Bacteriol. 190: 2266-2274, 2008). The enzyme has a high specificity to pyruvate as a substrate, and is known as typically being inhibited by isoleucine.

The terms “a protein having an activity of citramalate synthase” and “a gene encoding the same” in the present disclosure may include any proteins having an activity of citramalate synthase as described above and any genes encoding the same without limitation.

Specifically, a protein having an activity of citramalate synthase may be derived from Methanocaldococcus, and more specifically, Methanocaldococcus jannaschii. The citramalate synthase has the amino acid sequence of SEQ ID NO: 1, and cimA gene encoding the same has a nucleotide sequence of SEQ ID NO: 2. Amino acid sequences of a protein showing the activity are different depending on the species or strain of microorganism, or multiple nucleic acids having an identical function may encode any predetermined proteins because of degeneracy of a genetic code, and accordingly, the sequences are not limited to the SEQ ID NOS described above.

Further, any proteins substantially having an activity of citramalate synthase may be included in the scope of the present disclosure without limitation as long as the protein substantially has an activity of citramalate synthase as an amino acid sequence showing 70% or more homology to SEQ ID NO: 1, specifically 80% or more homology thereto, more specifically 90% or more homology thereto, more specifically 95% or more homology thereto, and even more specifically 98% or more homology thereto.

Additionally, if an amino acid sequence with the homology to the sequences substantially has a biological activity identical or corresponding to a protein of SEQ ID NO: 1, an amino acid sequence whose portion is deleted, modified, substituted, or added is also obviously included in the scope of the present disclosure.

Further, a gene encoding the citramalate synthase may have a nucleotide sequence encoding the amino acid sequence of SEQ ID NO: 1. Herein, because of the degeneracy of a codon or considering a codon preferred by an organism to express the protein, a polynucleotide may have various transformations in the encoding regions within the scope where an amino acid sequence of the protein is not changed. The polynucleotide sequence may, for example, have the polynucleotide sequence of SEQ ID NO: 2, and a nucleotide sequence having 80% or more homology thereto, specifically 90% or more homology thereto, and more specifically 99% or more homology thereto, but is not limited thereto.

Further, in order to recover the mutant having an enhanced activity of citramalate synthase, the present disclosure provides a mutant library of CimA(M) recovered using error-prone PCR as a mutant of a protein of SEQ ID NO: 1.

Specifically, the mutant library of CimA(M) of the present disclosure may include the following:

CimA(M)m1 having the amino acid sequence of SEQ ID NO: 3 (specifically, possibly being encoded from a polynucleotide having a nucleotide sequence of SEQ ID NO: 4);

CimA(M)m2 having the amino acid sequence of SEQ ID NO: 5 (specifically, possibly being encoded from a polynucleotide having a nucleotide sequence of SEQ ID NO: 6);

CimA(M)m3 having the amino acid sequence of SEQ ID NO: 7 (specifically, possibly being encoded from a polynucleotide having a nucleotide sequence of SEQ ID NO: 8);

CimA(M)m4 having the amino acid sequence of SEQ ID NO: 9 (specifically, possibly being encoded from a polynucleotide having a nucleotide sequence of SEQ ID NO: 10);

CimA(M)m5 having the amino acid sequence of SEQ ID NO: 11 (specifically, possibly being encoded from a polynucleotide having a nucleotide sequence of SEQ ID NO: 12);

CimA(M)m6 having the amino acid sequence of SEQ ID NO: 13 (specifically, possibly being encoded from a polynucleotide having a nucleotide sequence of SEQ ID NO: 14); and

CimA(M)m7 having the amino acid sequence of SEQ ID NO: 15 (specifically, possibly being encoded from a polynucleotide having a nucleotide sequence of SEQ ID NO: 16).

Further, any proteins substantially having an activity of a citramalate synthase mutant may be included in the scope of the present disclosure without limitation as long as the protein substantially has an activity of the citramalate synthase mutant as an amino acid sequence showing a homology to amino acid sequences of the SEQ ID NOS of specifically 80% or more, more specifically 90% or more, even more specifically 95% or more, and far even more specifically 99% or more.

The term “homology” in the present disclosure refers to a degree of similarity to a given amino acid or nucleotide sequence, and may be represented as a percentage. In the present disclosure, homology of a sequence having an activity identical or similar to a given amino acid or nucleotide sequence is represented as “% homology”. For example, homology may be identified using standard software, specifically BLAST 2.0, for calculating parameters such as score, identity, and similarity or by comparing sequences through Southern hybridization under defined stringent conditions. Appropriate hybridization conditions may be defined within the scope of the art (e.g., J. Sambrook et al., Molecular Cloning, A Laboratory Manual, 1989), and may be determined using methods well known by one of ordinary skill in the art.

In a specific embodiment, a microorganism of the present disclosure may include citramalate synthase of SEQ ID NO: 1 derived from Methanocaldococcus jannaschii, a mutant thereof, and a gene encoding a protein of an amino acid sequence selected from the group consisting of SEQ ID NOS: 1, 3, 5, 7, 9, 11, 13, and 15. More specifically, a desired gene may be expressed by transforming recombinant vectors including each gene into the microorganism.

The term “recombinant vector” in the present disclosure refers to a DNA construct containing a nucleotide sequence of a polynucleotide encoding a desired protein, which is operably linked to an appropriate regulatory sequence to express the desired protein in a suitable host. The regulatory sequence includes a promoter that can initiate transcription, an optional operator sequence for regulating such transcription, a sequence encoding a suitable mRNA ribosome binding site, and a sequence regulating the termination of transcription and translation. After the recombinant vector is transformed into a suitable host cell, it can replicate or function regardless of a genome of the host, and can be integrated into the genome itself.

A recombinant vector used in the present disclosure is not particularly limited as long as it is able to replicate in a host cell, and any vectors known in the art may be used. Examples of conventionally used vectors may include a natural or recombinant plasmid, cosmid, virus, and bacteriophage. For example, as a phage vector or cosmid vector, pWE15, M13, MBL3, MBL4, IXII, ASHII, APII, t10, t11, Charon4A, Charon21A, or the like may be used. As a plasmid vector, a pBR type, pUC type, pBluescriptII type, pGEM type, pTZ type, pCL type, pET type, or the like may be used. Vectors usable in the present disclosure are not particularly limited, and any known expression vectors can be used. Specifically, a pECCG117, pDZ, pACYC177, pACYC184, pCL, pUC19, pBR322, pMW118, or pCC1BAC vector, or the like may be used.

In an exemplary embodiment, a host cell may introduce a polynucleotide encoding a desired protein in the chromosome by a vector for chromosomal insertion. The introduction of the polynucleotide into the chromosome may be performed by any method known in the art, for example, homologous recombination, but is not limited thereto.

The term “transformation” in the present disclosure means that a recombinant vector containing a polynucleotide encoding a target protein is introduced into a host cell such that the protein encoded by the polynucleotide can be expressed in the host cell. The transformed polynucleotide may be any one as long as the polypeptide can be expressed in the host cell, regardless of whether the polynucleotide is inserted into or positioned outside a chromosome.

Additionally, the phrase “operably linked” refers to a functional connection between a promoter sequence, which initiates and mediates the transcription of the polynucleotide encoding the target protein of the present disclosure, and the gene sequence.

Meanwhile, in a specific embodiment of the present disclosure, a microorganism of the genus Corynebacterium may further comprise enhanced activities of 3-isopropylmalate dehydrogenase and 3-isopropylmalate dehydratase.

The phrase “enhancement of an activity of a protein” in the present disclosure refers to an activity of the protein enhanced compared with that of a wild-type or pre-modified protein. Specifically, the enhancement of an activity of a protein may include activity increases resulting from improvement of an activity of an endogenous gene encoding the protein, amplification of the endogenous gene by internal or external factors, deletion of a regulatory factor for suppressing the gene expression, an increase in the copy number of a gene, introduction of a foreign gene, modification of an expression regulatory sequence, in particular, replacement or modification of a promoter, and mutation within a gene.

Specifically, the enhancement of an activity of 3-isopropylmalate dehydrogenase or 3-isopropylmalate dehydratase may be performed by a method selected from the group consisting of:

1) increasing the copy number of a polynucleotide encoding said the protein;

2) modifying an expression regulatory sequence to increase the expression of the polynucleotide;

3) modifying the polynucleotide sequence on the chromosome to enhance the activity of the protein; deleting a regulatory factor for suppressing the gene expression; or

4) a combination thereof; however, the method is not particularly limited thereto.

The method of increasing the copy number of the polynucleotide may be performed in the form of operable linkage to a vector, or by insertion into a chromosome in a host cell; however, the method is not particularly limited thereto. Specifically, it may be performed by inserting a vector into a host cell, wherein the vector is operably linked to a polynucleotide encoding the protein of the present disclosure, and can replicate and function regardless of the host cell. Alternatively, it may be performed by a method for increasing the copy number of the polynucleotide inside a chromosome in a host cell by inserting a vector into the host cell, wherein the vector is operably linked to the polynucleotide and enables the polynucleotide to be inserted into the chromosome in the host cell.

The method of modifying an expression regulatory sequence to increase the expression of the polynucleotide may be performed by inducing a mutation on the sequence by deletion, insertion, or non-conservative or conservative substitution of a nucleic acid sequence, or a combination thereof, such that an activity of the expression regulatory sequence can be enhanced, or by replacing the sequence with a nucleic acid sequence having an enhanced activity; however, the method is not particularly limited thereto. The expression regulatory sequence includes a promoter, an operator sequence, a sequence encoding a ribosomal binding site, a sequence regulating the termination of transcription and translation, or the like, but is not particularly limited thereto.

A strong heterologous promoter instead of the original promoter may be linked upstream of a unit for the polynucleotide expression, and examples of the strong promoter may include cj7 promoter (Korean Patent No. 10-0620092 and International Publication No. WO 2006/065095), EF-Tu promoter, groEL promoter, aceA or aceB promoter, and more preferably, cj7 promoter as a Corynebacterium-derived promoter is operably linked so that an expression rate of the polynucleotide encoding the protein can be increased.

Additionally, the method of modifying the polynucleotide sequence on the chromosome may be performed by inducing a mutation on the expression regulatory sequence by deletion, insertion, or non-conservative or conservative substitution of a nucleic acid sequence, or a combination thereof, such that an activity of the polynucleotide sequence can be enhanced, or by replacing the sequence with a polynucleotide sequence modified to have an enhanced activity; however, the method is not particularly limited thereto.

The terms “3-isopropylmalate dehydrogenase” and “3-isopropylmalate dehydratase” in the present disclosure refer to enzymes involved in biosynthesis pathways of L-leucine, L-isoleucine, and L-valine. A protein having an activity of the enzymes and a gene encoding the same may include any proteins having an activity of the enzymes and any genes encoding the same without limitation.

Specifically, in the present disclosure, 3-isopropylmalate dehydrogenase (EC1.1.1.85) may be derived from a microorganism of the genus Corynebacterium, more specifically, from Corynebacterium glutamicum. Meanwhile, 3-isopropylmalate dehydrogenase in the present disclosure may have the amino acid sequence of SEQ ID NO: 17. Further, any protein substantially having an activity of 3-isopropylmalate dehydrogenase as an amino acid sequence substantially showing homology to SEQ ID NO: 17 of 70% or more, specifically 80% or more, more specifically 90% or more, even more specifically 95% or more, and far even more specifically 99% or more may be included in the scope of the present disclosure without limitation.

Further, a gene encoding the 3-isopropylmalate dehydrogenase may have a nucleotide sequence encoding the amino acid sequence of SEQ ID NO: 17. Amino acid sequences of a protein showing the activity are different depending on the species or strain of microorganism, or multiple nucleic acids having an identical function may encode any predetermined proteins because of degeneracy of a genetic code, and accordingly, the gene is not limited to the disclosed SEQ ID NOS. For example, the gene may have a nucleotide sequence of SEQ ID NO: 18, or a nucleotide sequence having homology to SEQ ID NO: 18 of 80%, specifically 90% or more, and more specifically 99% or more.

Additionally, 3-isopropylmalate dehydratase (EC4.2.1.33) in the present disclosure may be derived from a microorganism of the genus Corynebacterium, more specifically, from Corynebacterium glutamicum. Meanwhile, 3-isopropylmalate dehydratase in the present disclosure consists of two small/large subunits which may have the amino acid sequence of SEQ ID NOS: 19 and 21. Further, any proteins substantially having an activity of 3-isopropylmalate dehydratase may be included in the scope of the present disclosure without limitation as long as the protein substantially has an activity of 3-isopropylmalate dehydratase as an amino acid sequence showing homology to SEQ ID NOS: 19 and 21 of 70% or more, specifically 80% or more, more specifically 90% or more, even more specifically 95% or more, and far even more specifically 99% or more.

Additionally, a gene encoding the 3-isopropylmalate dehydratase may have a nucleotide sequence encoding amino acid sequences of SEQ ID NOS: 19 and 21. Amino acid sequences of a protein showing the activity are different depending on the species or strain of microorganism, or multiple nucleic acids having an identical function may encode any predetermined proteins because of degeneracy of a genetic code, and accordingly, the gene is not limited to the SEQ ID NOS. For example, the gene may have nucleotide sequences of SEQ ID NOS: 20 and 22, or nucleotide sequences having homology thereto of 80%, specifically 90% or more, and more specifically 99% or more.

Meanwhile, in a specific embodiment of the present disclosure, a microorganism of the genus Corynebacterium may include an enzyme involved in a biosynthesis pathway of L-isoleucine further including an enhanced activity.

In the present disclosure, the term “enzyme involved in a biosynthesis pathway of L-isoleucine” may include aspartate kinase (lysC gene), aspartate-β-semialdehyde dehydrogenase (asd gene), homoserine dehydrogenase (hom gene), homoserine kinase (thrB gene), threonine synthase (thrC gene), threonine dehydratase (ilvA gene), aminotransferase (ilvE gene), or the like, but is not limited thereto.

Meanwhile, in a specific embodiment of the present disclosure, a microorganism of the genus Corynebacterium may further include inactivated pyruvate dehydrogenase.

The term “pyruvate dehydrogenase (EC1.2.4.1)” in the present disclosure refers to an enzyme converting pyruvate to acetyl-CoA and CO₂. The present disclosure can prepare a strain with deleted aceE gene encoding the enzyme to increase the provision of acetyl-CoA used as a precursor of the cimA gene.

Specifically, pyruvate dehydrogenase (EC1.2.4.1) in the present disclosure may be derived from a microorganism of the genus Corynebacterium, and more specifically, from Corynebacterium glutamicum. Meanwhile, pyruvate dehydrogenase in the present disclosure may have the amino acid sequence of SEQ ID NO: 25. Further, any protein substantially having an activity of pyruvate dehydrogenase as an amino acid sequence substantially showing homology to SEQ ID NO: 25 of 70% or more, specifically 80% or more, more specifically 90% or more, even more specifically 95% or more, and far even more specifically 99% or more may be included in the scope of the present disclosure without limitation.

Additionally, a gene encoding the pyruvate dehydrogenase may have a nucleotide sequence encoding the amino acid sequence of SEQ ID NO: 25. Amino acid sequences of a protein showing the activity are different depending on the species or strain of microorganism, or multiple nucleic acids having an identical function may encode any predetermined proteins because of degeneracy of a genetic code, and accordingly, the gene is not limited to the disclosed SEQ ID NOS. For example, the gene may have the nucleotide sequence of SEQ ID NO: 26, or nucleotide sequences having homology thereto of 80%, specifically 90% or more, and more specifically 99% or more.

The term “inactivation” in the present disclosure refers to the cases in which a gene encoding the relevant polypeptide is not expressed, exhibits a decrease in the activity due to incomplete expression, or does not produce the relevant functional polypeptide, even though the gene is expressed.

Additionally, the term refers to a case in which a gene encoding the relevant polypeptide is substantially not expressed due to a significantly low expression level as well as a case in which the gene is completely inactivated. Accordingly, the inactivation of a gene may be complete (knock-out) or partial (e.g., a gene may be a hypomorph expressing a below-normal level or a product of a mutant gene showing a partial reduction in an activity affected by the gene).

Further, the “inactivation” of the relevant polypeptide in the present disclosure includes cases of elimination of an activity of the polypeptide as well as reduction thereof compared with an unmodified strain. Specifically, the inactivation of threonine dehydratase in the present disclosure may be performed by a method selected from the group consisting of:

1) a partial or complete deletion of a polynucleotide encoding the protein;

2) modification of an expression regulatory sequence to decrease the expression of the polynucleotide;

3) modification of the polynucleotide sequence on the chromosome to attenuate the activity of the protein; or

4) a combination thereof; however, the method is not particularly limited thereto.

The method of a partial or complete deletion of a polynucleotide encoding the protein may be performed by replacing a polynucleotide encoding an endogenous target protein in a chromosome with a marker gene or a polynucleotide in which a nucleic acid sequence was partially deleted, through a vector for chromosomal insertion into a bacterium. As used herein, the term “partially” may vary depending on types of polynucleotide, but may specifically refer to 1 to 300 nucleotides, more specifically, 1 to 100 nucleotides, and even more specifically, 1 to 50 nucleotides.

The method of modification of an expression regulatory sequence to decrease the expression of the polynucleotide may be performed by inducing a mutation on the sequence by deletion, insertion, or non-conservative or conservative substitution of a nucleic acid sequence, or a combination thereof, such that an activity of the expression regulatory sequence can be attenuated, or by replacing the sequence with a nucleic acid sequence having an attenuated activity; however, the method is not particularly limited thereto. The expression regulatory sequence includes a promoter, an operator sequence, a sequence encoding a ribosomal binding site, a sequence regulating the termination of transcription and translation, or the like; but is not limited thereto.

Further, the method of modification of the polynucleotide sequence on the chromosome may be performed by inducing a mutation on the sequence by deletion, insertion, or non-conservative or conservative substitution of a polynucleotide sequence, or a combination thereof, or by replacing the sequence with a polynucleotide sequence modified to have an attenuated activity; however, the method is not limited thereto.

In another specific embodiment of the present disclosure, any microorganism as a microorganism for producing L-isoleucine may be included in the scope of the present disclosure without limitation, as long as the microorganism can be expressed by introducing a protein having an activity of citramalate synthase. Examples of the microorganism may be a microorganism of the genus Escherichia, Shigella, Citrobacter, Salmonella, Enterobacter, Yersinia, Klebsiella, Erwinia, Corynebacterium, Brevibacterium, Lactobacillus, Selenomanas, Vibrio, Pseudomonas, Streptomyces, Arcanobacterium, Alcaligenes, or the like, and specifically the genus Corynebacterium, more specifically, Corynebacterium glutamicum, but are not limited thereto.

Still another aspect of the present disclosure provides a method for producing L-isoleucine comprising

culturing a microorganism of the genus Corynebacterium comprising a protein having an activity of citramalate synthase in a medium; and

recovering L-isoleucine from the microorganism or medium,

wherein a microorganism of the genus Corynebacterium having an introduced gene encoding a protein having an activity of citramalate synthase is as described above.

Briefly, the microorganism of the genus Corynebacterium may have an introduced gene encoding citramalate synthase derived from Methanocaldococcus, wherein the citramalate synthase may specifically have an amino acid sequence selected from the group consisting of SEQ ID NOS: 1, 3, 5, 7, 9, 11, 13, and 15.

Additionally, the microorganism of the genus Corynebacterium in the present disclosure may further include enhanced activities of 3-isopropylmalate dehydrogenase and 3-isopropylmalate dehydratase. Further, the microorganism of the genus Corynebacterium may further include inactivated pyruvate dehydrogenase.

Specifically, a microorganism of the genus Corynebacterium in an example of the present disclosure may be Corynebacterium glutamicum having an introduced gene encoding citramalate synthase.

In the method, culturing a microorganism of the genus Corynebacterium according to the present disclosure may be performed by a well-known batch culture, continuous culture, and fed-batch culture, but is not particularly limited thereto.

In particular, for the culture conditions, an appropriate pH (e.g., a pH of 5 to 9, and specifically, a pH of 6 to 8) may be adjusted using a basic compound (e.g., sodium hydroxide, potassium hydroxide, or ammonia) or an acidic compound (e.g., phosphoric acid or sulfuric acid), but is not particularly limited thereto. In addition, bubble formation may be inhibited using an antifoaming agent, such as fatty acid polyglycol ester.

Oxygen or an oxygen-containing gas mixture may be introduced into the culture to maintain aerobic conditions, and the temperature of the culture is usually 20° C. to 45° C., specifically, 25° C. to 40° C. The cultivation continues until the maximum amount of L-isoleucine produced is obtained, and may usually take 10 hours to 160 hours to achieve such objective. L-Isoleucine may be secreted into the medium or may remain in the cells.

Further, as a carbon source for a culture medium used, sugars and carbohydrates (e.g., glucose, sucrose, lactose, fructose, maltose, molasses, starch, and cellulose), oils and fats (e.g., soybean oil, sunflower seed oil, peanut oil, and coconut oil), fatty acids (e.g., palmitic acid, stearic acid, and linoleic acid), alcohols (e.g., glycerol and ethanol), organic acids (e.g., acetic acid), or the like may be used individually or in combination, but the carbon source is not limited thereto.

As a nitrogen source, nitrogen-containing organic compounds (e.g., peptone, yeast extract, meat juice, malt extract, corn precipitate, soybean meal powder, and urea), inorganic compounds (e.g., ammonium sulfate, ammonium chloride, ammonium phosphate, ammonium carbonate, and ammonium nitrate), or the like may be used individually or in combination, but the nitrogen source is not limited thereto.

As a phosphorous source, potassium dihydrogen phosphate, dipotassium hydrogen phosphate, sodium-containing salts corresponding thereto, or the like may be used individually or in combination, but the phosphorous source is not limited thereto.

In a specific embodiment of the present disclosure, upon culturing a microorganism of the genus Corynebacterium in the method for producing L-isoleucine, acetate may be further provided.

For a method for recovering L-isoleucine produced by culturing the microorganism in the method, an appropriate method may be used to recover the target amino acid from the culture fluid depending on a culturing method known in the art, e.g., batch culture, continuous culture, fed-batch culture, etc. For example, centrifugation, filtration, anion exchange chromatography, crystallization, HPLC, or the like may be used, but a method for recovering L-isoleucine is not limited thereto.

Still another aspect of the present disclosure provides the use of a microorganism of the genus Corynebacterium comprising a protein having an activity of citramalate synthase for producing L-isoleucine.

In the use, the microorganism of the genus Corynebacterium having an introduced gene encoding a protein having an activity of citramalate synthase is as described above.

As described above, the microorganism of the genus Corynebacterium for producing L-isoleucine of the present disclosure can produce a high yield of L-isoleucine through a novel biosynthesis pathway which does not use L-threonine as a precursor by having an activity of citramalate synthase introduced.

### DETAILED DESCRIPTION OF EMBODIMENT

### Example 1: Construction of a Recombinant Vector Including cimA Gene Derived from Methanocaldococcus

<1-1> Preparation of a Fragment of cimA Gene Derived from Methanocaldococcus

To obtain a 1476 bp fragment comprising the open reading frame (ORF) of cimA gene (NC_000909.1) encoding citramalate synthase, genomic DNA of Methanocaldococcus jannaschii DSM 2661 was extracted using a Genomic-tip system (Qiagen).

Citramalate synthase derived from Methanocaldococcus jannaschii DSM 2661 has the sequence of 491 amino acids represented by SEQ ID NO: 1, and cimA gene encoding the same has the nucleotide sequence of SEQ ID NO: 2.

Polymerase chain reaction (hereinafter abbreviated as “PCR”) was performed using the extracted genomic DNA (gDNA) as a template. The PCR was performed using the primer pair of SEQ ID NOS: 35 and 36 under the following conditions: 30 cycles, each consisting of denaturation at 95° C. for 30 sec, annealing at 56° C. for 30 sec, and elongation at 72° C. for 60 sec.

The amplified PCR product was electrophoresed on 1.0% agarose gel, then a band having a desired size was eluted for recovery, and the product was labeled as “cimA(M) fragment”.

primer cimA-5-NdeI (SEQ ID NO: 35):
5′-GCATCATATGATGGTAAGGATATTC-3′
primer cimA-3-XbaI (SEQ ID NO: 36):
5′-CGATCTAGATTAATTCAACAACATGTT-3′

<1-2> Construction of Recombinant Vector p117-Cj7-cimA(M)

PCR was performed using p117-cj7-gfp including promoter cj7 derived from a known microorganism of the genus Corynebacterium (Korean Patent No. 10-0620092) as a template. As used herein, the term “p117” represents pECCG117, which is an E. coli-Corynebacterium shuttle vector (Biotechnology Letters 13(10): 721-726, 1991). The PCR was performed using the primer pair of SEQ ID NOS: 27 and 28 under the following conditions: 30 cycles, each consisting of denaturation at 95° C. for 30 sec, annealing at 56° C. for 20 sec, and elongation at 72° C. for 30 sec.

The amplified PCR product was electrophoresed on 1.0% agarose gel, then a 323 bp band was eluted for recovery, and the product was labeled as “cj7 fragment”.

primer Pcj7-5-KpnI (SEQ ID NO: 27):
5′-GATGGTACCACCCCAGAAACATCCCAGC-3′
primer Pcj7-3 NdeI (SEQ ID NO: 28):
5′-CGATCATATGGAGTGTTTCCTTTCGTTGGG-3′

Sewing PCR was performed using the prepared cj7 fragment and cimA(M) fragment prepared in Example <1-1> above as a template. The sewing PCR was performed using the primer pair of SEQ ID NOS: 27 and 36 under the following conditions: 30 cycles, each consisting of denaturation at 95° C. for 30 sec, annealing at 56° C. for 30 sec, and elongation at 72° C. for 60 sec.

The amplified PCR product was electrophoresed on 1.0% agarose gel, then a 1799 bp band was eluted for recovery, and the product was labeled as “cj7-cimA(M) fragment”.

The obtained cj7-cimA(M) fragment was treated with restriction enzymes KpnI and XbaI, and then ligated with a linear p117 fragment treated with the same restriction enzymes.

E. coli DH5a cells were heat-shock transformed with the constructed vector, then plated on a 25 μg/mL kanamycin-containing LB solid medium, and cultured overnight at 37° C. One platinum loop of the cultured colony was inoculated into 3 mL of a 25 μg/mL kanamycin-containing LB liquid medium and cultured overnight, and then plasmid DNA was recovered using a plasmid miniprep kit (Catalogue No. 27104, Qiagen, hereinafter the same).

The construction of the recombinant vector was confirmed by treatment with restriction enzymes KpnI and XbaI, and the clone was identified by performing PCR using the primer pair of SEQ ID NOS: 29 and 30 under the following conditions: 30 cycles, each consisting of denaturation at 95° C. for 30 sec, annealing at 56° C. for 30 sec, and elongation at 72° C. for 90 sec. The recovered recombinant vector was labeled as “p117-cj7-cimA(M)”.

primer 117-F (SEQ ID NO: 29):
5′-CCACAGCCGACAGGATGGTGA-3′
primer 117-R (SEQ ID NO: 30):
5′-CTCAGGGTGTAGCGGTTCGGT-3′

<1-3> Preparation of a Fragment of leuBCD Gene Involved in the Biosynthesis Pathway of 2-Ketobutyrate

To prepare a recombinant vector for enhancing a gene in the biosynthesis pathway of 2-ketobutyrate, a fragment of the leuBCD gene encoding 3-isopropylmalate dehydrogenase and 3-isopropylmalate dehydratase derived from a microorganism of the genus Corynebacterium was prepared as described below.

To obtain a 1359 bp fragment comprising the ORF of leuB gene encoding 3-isopropylmalate dehydrogenase (EC1.1.1.85), genomic DNA of Corynebacterium glutamicum ATCC13032 was extracted using a Genomic-tip system (Qiagen).

3-Isopropylmalate dehydrogenase derived from Corynebacterium glutamicum ATCC13032 has the sequence of 340 amino acids represented by of SEQ ID NO: 17, and leuB gene encoding the same has the nucleotide sequence of SEQ ID NO: 18.

PCR was performed using the extracted gDNA as a template. The PCR was performed using the primer pair of SEQ ID NOS: 31 and 32 under the following conditions: 30 cycles, each consisting of denaturation at 95° C. for 30 sec, annealing at 56° C. for 30 sec, and elongation at 72° C. for 60 sec.

The amplified PCR product was electrophoresed on 1.0% agarose gel, then a band having a desired size was eluted for recovery, and the product was labeled as “leuB fragment”.

primer leuB-5-cimA (SEQ ID NO: 31):
5′-TTGTTGAATTAATCTAGAGGTGACACCCCAGTGG-3′
primer leuB-3-leuC (SEQ ID NO: 32):
5′-TCGCAGCTGCCACCGATATTTAGCTTTGCAGCGC-3′

To obtain a 2078 bp fragment comprising the ORF of leuCD gene encoding 3-isopropylmalate dehydratase (EC4.2.1.33), PCR was performed using gDNA of Corynebacterium glutamicum ATCC13032 as a template.

LeuC gene encodes a large subunit of 3-isopropylmalate dehydratase, and the leuC gene derived from Corynebacterium glutamicum ATCC13032 has the nucleotide sequence of SEQ ID NO: 20 encoding a polypeptide having the amino acid sequence of SEQ ID NO: 19. Further, leuD gene encodes a small subunit of 3-isopropylmalate dehydratase, and the leuD gene derived from Corynebacterium glutamicum ATCC13032 has the nucleotide sequence of SEQ ID NO: 22 encoding a polypeptide having the amino acid sequence of SEQ ID NO: 21.

The PCR was performed using the primer pair of SEQ ID NOS: 33 and 34 under the following conditions: 30 cycles, each consisting of denaturation at 95° C. for 30 sec, annealing at 56° C. for 30 sec, and elongation at 72° C. for 80 sec.

The amplified PCR product was electrophoresed on 1.0% agarose gel, then a band having a desired size was eluted for recovery, and the product was labeled as “leuCD fragment”.

primer leuC-5-leuB (SEQ ID NO: 33):
5′-GCGCTGCAAAGCTAAATATCGGTGGCAGCTGCGA-3′
primer leuD-3-XbaI (SEQ ID NO: 34):
5′-TGGCGGCCGCTCTAGAGCTTTCGCTATCAGACTG-3′

Sewing PCR was performed using the leuB fragment and leuCD fragment recovered above as a template. The sewing PCR was performed using the primer pair of SEQ ID NOS: 31 and 34 under the following conditions: 30 cycles, each consisting of denaturation at 95° C. for 30 sec, annealing at 56° C. for 30 sec, and elongation at 72° C. for 120 sec.

The amplified PCR product was electrophoresed on 1.0% agarose gel, then a 3437 bp band was eluted for recovery, and the product was labeled as “leuBCD fragment”.

<1-4> Construction of Recombinant Vector p117-Cj7-cimA(M)-leuBCD

To prepare a recombinant vector for enhancing a gene in the biosynthesis pathway of 2-ketobutyrate, p117-cj7-cimA(M), which is the recombinant vector constructed in Example <1-2> above, was treated with restriction enzyme XbaI, the leuBCD fragment recovered in Example <1-3> above was treated with restriction enzyme XbaI, and then both fragments were ligated.

E. coli DH5α cells were heat-shock transformed with the constructed vector, then plated on a 25 μg/mL kanamycin-containing LB solid medium, and cultured overnight at 37° C. One platinum loop of the cultured colony was inoculated into 3 mL of a 25 μg/mL kanamycin-containing LB liquid medium and cultured overnight, and then plasmid DNA was recovered using a plasmid miniprep kit.

The construction of the recombinant vector was confirmed by treatment with restriction enzyme XbaI, and the clone was identified by performing PCR using the primer pair of SEQ ID NOS: 29 and 30 under the following conditions: 30 cycles, each consisting of denaturation at 95° C. for 30 sec, annealing at 56° C. for 30 sec, and elongation at 72° C. for 180 sec. The recovered recombinant vector was labeled as “p117-cj7-cimA(M)-leuBCD”.

<1-5> Preparation of a Fragment of a cimA(M)Mutant Using Error-Prone PCR

To secure a DNA pool having a random mutation introduced to cimA gene, PCR was performed using the cimA(M) fragment recovered in Example <1-1> above as a template and the diversify PCR random mutagenesis kit (Catalogue No. 630703, Clonetech). The PCR was performed on condition 4 of the mutagenesis reaction in Table III in the user manual of the product using the primer pair of SEQ ID NOS: 35 and 36 under the following conditions: 25 cycles, each consisting of denaturation at 95° C. for 30 sec and elongation at 68° C. for 30 sec.

As a result of the PCR, a mutated art cimA DNA pool having a modification of nucleotide substitution randomly introduced was prepared. The PCR product (hereinafter, referred to as “cimA(M)m fragment”) was electrophoresed on 1.0% agarose gel then eluted for recovery.

primer cimA-5-NdeI (SEQ ID NO: 35):
5′-GCATCATATGATGGTAAGGATATTC-3′
primer cimA-3-XbaI (SEQ ID NO: 36):
5′-CGATCTAGATTAATTCAACAACATGTT-3′

<1-6> Construction of Mutant Library of p117-Cj7-cimA(M)m

Recombinant vector, p117-cj7-cimA(M)m, constructed in Example <1-2> above, was treated with restriction enzymes NdeI and XbaI, and was ligated together with the cimA(M)m fragment recovered in Example <1-5> above and treated with the same restriction enzymes.

E. coli DH5a cells were heat-shock transformed with the constructed vector, then plated on a 25 μg/mL kanamycin-containing LB solid medium, and cultured overnight at 37° C. The cultured colonies were gathered, and then plasmid DNA was recovered using a plasmid miniprep kit thereby construct the “mutant library of p117-cj7-cimA(M)m”.

### Example 2: Confirmation of L-Isoleucine Producing Ability by Introduction of cimA Gene

To confirm whether Corynebacterium glutamicum can has L-isoleucine producing ability when cimA gene derived from Methanocaldococcus is introduced thereto, a strain having ilvA gene removed encoding threonine dehydratase was constructed, and recovery of the L-isoleucine producing ability of the strain was examined by introducing the cimA gene thereto.

Threonine dehydratase is an enzyme converting L-threonine, which is a precursor of L-isoleucine biosynthesis, to 2-ketobutyrate. Threonine dehydratase derived from Corynebacterium glutamicum ATCC13032 has the amino acid sequence of SEQ ID NO: 23, and ilvA gene encoding the same has the nucleotide sequence of SEQ ID NO: 24. Further, any protein having an activity of threonine dehydratase is included in the scope of the present disclosure without limitation.

<2-1> Construction of ilvA Gene-Deleted Vector

To obtain an ilvA-gene fragment, PCR was performed using genomic DNA extracted from Corynebacterium glutamicum ATCC13032 using a Genomic-tip system (Qiagen) as a template. The PCR was performed using each primer pair of SEQ ID NOS: 37 and 38, and SEQ ID NOS: 39 and 40 under the following conditions: 30 cycles, each consisting of denaturation at 95° C. for 30 sec, annealing at 56° C. for 30 sec, and elongation at 72° C. for 45 sec.

The amplified PCR products were electrophoresed on 1.0% agarose gel, and then 547 bp and 467 bp bands for each PCR were eluted for recovery.

Sewing PCR was performed using the two fragments of the ilvA gene recovered above as a template. The sewing PCR was performed using the primer pair of SEQ ID NOS: 37 and 40 under the following conditions: 30 cycles, each consisting of denaturation at 95° C. for 30 sec, annealing at 56° C. for 30 sec, and elongation at 72° C. for 60 sec.

The amplified PCR product was electrophoresed on 1.0% agarose gel, then a 1014 bp band was eluted for recovery, and the recovered ilvA gene-deleted fragment was labeled as “DilvA”.

The DilvA fragment recovered above was treated with restriction enzyme XbaI, pDZ vector (Korean Patent No. 10-0924065) was treated with same restriction enzyme to recover a linear pDZ fragment, and then the DilvA and linear pDZ fragments were ligated to construct a recombinant vector having ilvA gene deleted.

E. coli DH5α cells were heat-shock transformed with the constructed recombinant vector, then plated on a 25 μg/mL kanamycin-containing LB solid medium, and cultured overnight at 37° C. One platinum loop of the cultured colony was inoculated into 3 mL of a 25 μg/mL kanamycin-containing LB liquid medium and cultured overnight, and then plasmid DNA was recovered using a plasmid miniprep kit.

The construction of the recombinant vector was confirmed by treatment with restriction enzyme XbaI, and the clone was identified by performing PCR using the primer pair of SEQ ID NOS: 41 and 42 under the following conditions: 30 cycles, each consisting of denaturation at 95° C. for 30 sec, annealing at 56° C. for 30 sec, and elongation at 72° C. for 90 sec. The recovered recombinant vector was labeled as “pDZ-ilvA(Del)”.

primer ilvA-5-XbaI (SEQ ID NO: 37):
5′-GCATCTAGAGAACACGGACAATGCCAC-3′
primer ilvA-Del-R (SEQ ID NO: 38):
5′-CAAACAGCGTGATGTCCTGAGTGAGCTGCGCT-3′
primer ilvA-Del-F (SEQ ID NO: 39):
5′-AGCGCAGCTCACTCAGGACATCACGCTGTTTG-3′
primer ilvA-3-XbaI (SEQ ID NO: 40):
5′-GCATCTAGAACCTGCGGCACACCTTGGGC-3′
primer Topo-F (SEQ ID NO: 41):
5′-GCAGTGAGCGCAACGCAAT-3′
primer Topo-R (SEQ ID NO: 42):
5′-CGTTGTAAAACGACGGCCA-3′

<2-2> Construction of ilvA Gene-Deleted Strain

The recombinant vector pDZ-ilvA(Del) constructed in Example <2-1> above was introduced into Corynebacterium glutamicum ATCC13032 as a parent strain by electroporation, and was plated on a solid medium containing 25 μg/mL of kanamycin to select a single colony. From the colony, each strain having the pDZ-ilvA(Del) vector introduced into a chromosome was selected by the second passage, and then PCR was performed using gDNA recovered from each of the selected strains as a template. The PCR was performed using the primer pair of SEQ ID NOS: 43 and 44 under the following conditions: 30 cycles, each consisting of denaturation at 95° C. for 30 sec, annealing at 56° C. for 30 sec, and elongation at 72° C. for 90 sec.

As a result of the PCR, an ilvA gene-deleted strain was recovered, and was labeled as an “ATCC13032ΔilvA” strain.

primer CFilvA (SEQ ID NO: 43):
5′-GATCTGTGATGAGGTGATG-3′
primer CRilvA (SEQ ID NO: 44):
5′-CGTCCTTCCATGACTCTCA-3′

<2-3> Preparation of a Gene Fragment Involved in the Biosynthesis Pathway of L-Isoleucine

To prepare a recombinant vector for enhancing a gene in the biosynthesis pathway of L-isoleucine, fragments of lysC, asd, hom, thrB, thrC, and ilvA genes derived from a microorganism of the genus Corynebacterium were prepared as described below.

First, to obtain a DNA fragment comprising the ORF of lysC-asd gene encoding aspartate kinase and aspartate-β-semialdehyde dehydrogenase, PCR was performed using genomic DNA extracted from Corynebacterium glutamicum ATCC13032 as a template as described in Example <2-1>. The PCR was performed using the primer pair of SEQ ID NOS: 45 and 46 under the following conditions: 30 cycles, each consisting of denaturation at 95° C. for 30 sec, annealing at 56° C. for 30 sec, and elongation at 72° C. for 90 sec.

The amplified PCR product was electrophoresed on 1.0% agarose gel, then a 2666 bp band was eluted for recovery, and the product was labeled as “lysC-asd fragment”.

primer lysC-5-KpnI (SEQ ID NO: 45):
5′-TGAGGTACCCATGCGATTGTTAATGC-3′
primer asd-3-SfoI (SEQ ID NO: 46):
5′-CTAGGCGCCAGTGTAGCACTCAAGCGGA-3′

To obtain a DNA fragment comprising the ORF of hom-thrB gene encoding homoserine dehydrogenase and homoserine kinase, PCR was performed using genomic DNA extracted from Corynebacterium glutamicum ATCC13032 as a template as described in Example <2-1>. The PCR was performed using the primer pair of SEQ ID NOS: 47 and 48 under the following conditions: 30 cycles, each consisting of denaturation at 95° C. for 30 sec, annealing at 56° C. for 30 sec, and elongation at 72° C. for 90 sec.

The amplified PCR product was electrophoresed on 1.0% agarose gel, then a 2633 bp band was eluted for recovery, and the product was labeled as “hom-thrB fragment”.

primer hom-5-BamHI (SEQ ID NO: 47):
5′-CTAGGATCCCTCACCATTCTCAATGGT-3′
primer thrB-3-BamHI (SEQ ID NO: 48):
5′-CTAGGATCCGCCTTCCTTGTTGGGC-3′

To obtain a DNA fragment comprising the ORF of thrC gene encoding threonine synthase, PCR was performed using genomic DNA extracted from Corynebacterium glutamicum ATCC13032 as a template as described in Example <2-1>. The PCR was performed using the primer pair of SEQ ID NOS: 49 and 50 under the following conditions: 30 cycles, each consisting of denaturation at 95° C. for 30 sec, annealing at 56° C. for 30 sec, and elongation at 72° C. for 60 sec.

The amplified PCR product was electrophoresed on 1.0% agarose gel, then a 1703 bp band was eluted for recovery, and the product was labeled as “thrC fragment”.

primer thrC-5-SpeI (SEQ ID NO: 49):
5′-TGCACTAGTGAGAACATACAGGTTCCA-3′
primer thrC-3-ilvA (SEQ ID NO: 50):
5′-GGCATTGTCCGTGTTCTTACTTCACGGAAGTG-3′

To obtain a DNA fragment comprising the ORF of ilvA gene encoding threonine dehydratase, PCR was performed using genomic DNA extracted from Corynebacterium glutamicum ATCC13032 as a template as described in Example <2-1>. The PCR was performed using the primer pair of SEQ ID NOS: 51 and 52 under the following conditions: 30 cycles, each consisting of denaturation at 95° C. for 30 sec, annealing at 56° C. for 30 sec, and elongation at 72° C. for 60 sec.

The amplified PCR product was electrophoresed on 1.0% agarose gel, then a 1862 bp band was eluted for recovery, and the product was labeled as “ilvA fragment”.

primer ilvA-5-thrC (SEQ ID NO: 51):
5′-CACTTCCGTGAAGTAAGAACACGGACAATGCC-3′
primer ilvA-3-SpeI (SEQ ID NO: 52):
5′-TGCACTAGTACCTGCGGCACACCTTGGGC-3′

Sewing PCR was performed using the thrC fragment and ilvA fragment recovered above as a template. The sewing PCR was performed using the primer pair of SEQ ID NOS: 49 and 52 under the following conditions: 30 cycles, each consisting of denaturation at 95° C. for 30 sec, annealing at 56° C. for 30 sec, and elongation at 72° C. for 120 sec.

The amplified PCR product was electrophoresed on 1.0% agarose gel, then a 3565 bp band was eluted for recovery, and the product was labeled as “thrC-ilvA fragment”.

<2-4> Construction of a Vector for Enhancing a Gene in the Biosynthesis Pathway of L-Isoleucine

To prepare a recombinant vector for enhancing a gene in the biosynthesis pathway of L-isoleucine, the lysC-asd fragment obtained in Example <2-3> above was treated with restriction enzymes KpnI and SfoI, and then ligated with a linear p117 fragment treated with restriction enzymes KpnI and EcoRV.

A plasmid DNA was recovered in the same manner of Example <1-2> above. To confirm the construction of the recombinant vector, the clone was identified by performing PCR using the primer pair of SEQ ID NOS: 29 and 30 under the following conditions: 30 cycles, each consisting of denaturation at 95° C. for 30 sec, annealing at 56° C. for 30 sec, and elongation at 72° C. for 100 sec. The recovered recombinant vector was labeled as “p117-lysC-asd”.

The hom-thrB fragment obtained in Example <2-3> above was treated with restriction enzyme BamHI, and then ligated with a linear p117-lysC-asd fragment treated with the same restriction enzyme.

A plasmid DNA was recovered in the same manner of Example <1-2> above. To confirm the construction of the recombinant vector, the clone was identified by performing PCR using the primer pair of SEQ ID NOS: 29 and 30 under the following conditions: 30 cycles, each consisting of denaturation at 95° C. for 30 sec, annealing at 56° C. for 30 sec, and elongation at 72° C. for 180 sec. The recovered recombinant vector was labeled as “p117-lysC-asd-hom-thrB”.

The thrC-ilvA fragment obtained in Example <2-3> above was treated with restriction enzyme SpeI, and then ligated with a linear p117-lysC-asd-hom-thrB fragment treated with the same restriction enzyme.

A plasmid DNA was recovered in the same manner of Example <1-2> above. To confirm the construction of the recombinant vector, the clone was identified by performing PCR using the primer pair of SEQ ID NOS: 29 and 30 under the following conditions: 30 cycles, each consisting of denaturation at 95° C. for 30 sec, annealing at 56° C. for 30 sec, and elongation at 72° C. for 300 sec.

The recovered recombinant vector was labeled as “p117-IBGC (Isoleucine Biosynthesis Genes Cluster)”, which is a recombinant vector for enhancing a gene in the biosynthesis pathway of L-isoleucine including fragments of lysC, asd, hom, thrB, thrC, and ilvA genes, six genes involved in the biosynthesis of L-isoleucine.

<2-5> Comparison of L-Isoleucine Productivity

Recombinant vectors p117-cj7-cimA(M), p117-cj7-cimA(M)-leuBCD, and p117-IBGC constructed in Examples <1-2>, <1-4>, and <2-4> above were introduced into Corynebacterium glutamicum ATCC13032 by electroporation, and were plated on a solid medium containing 25 μg/mL of kanamycin to select a single colony. From the colony, each of the selected strains was labeled as Corynebacterium glutamicum “ATCC13032/p117-cj7-cimA(M)”, “ATCC13032/p117-cj7-cimA(M)-leuBCD”, and “ATCC13032/p117-IBGC”.

Further, p117-cj7-cimA(M) and p117-cj7-cimA(M)-leuBCD were introduced into a Corynebacterium glutamicum ATCC13032ΔilvA strain prepared in Example <2-2> above by electroporation, and were plated on a solid medium containing 25 μg/mL of kanamycin to select a single colony. From the colony, each of the selected strains was labeled as Corynebacterium glutamicum “ATCC13032ΔilvA/p117-cj7-cimA(M)” and “ATCC13032ΔilvA/p117-cj7-cimA(M)-leuBCD”.

Titer evaluation on L-isoleucine productivity was performed using Corynebacterium glutamicum ATCC13032, ATCC13032/p117-IBGC, ATCC13032/p117-cj7-cimA(M), ATCC13032/p117-cj7-cimA(M)-leuBCD, ATCC13032ΔilvA, ATCC13032ΔilvA/p117-cj7-cimA(M), and ATCC13032ΔilvA/p117-cj7-cimA(M)-leuBCD.

Specifically, each of the strains was inoculated into a 250 mL flask containing 25 mL of a glucose-containing titer medium having the composition shown in Table 1 below, and was then cultured in an incubator at 32° C. and 200 rpm for 30 hours. The concentrations of L-isoleucine produced are shown in Table 2 below.

TABLE 1

| Composition | Concentration (per liter) | |
|---|---|---|
| Glucose | 60 | g |
| KH₂PO₄ | 1.1 | g |
| (NH₄)₂SO₄ | 20 | g |
| MgSO₄•7H₂O | 1.2 | g |
| FeSO₄•7H₂O | 90 | mg |
| MnSO₄•4H₂O | 90 | mg |
| Thiamine-HCl | 4.5 | mg |
| d-Biotin | 0.9 | mg |
| HSM | 20 | g |
| Calcium carbonate | 30 | g |

| | |
|---|---|
| pH | 7.0 |

TABLE 2

| Strain | L-isoleucine (g/L) |
|---|---|
| ATCC13032 | 0.02 |
| ATCC13032/p117-IBGC | 0.70 |
| ATCC13032/p117-cj7-cimA(M) | 0.83 |
| ATCC13032/p117-cj7-cimA(M)-leuBCD | 1.25 |
| ATCC13032ΔilvA | 0.00 |
| ATCC13032ΔilvA/p117-cj7-cimA(M) | 0.08 |
| ATCC13032ΔilvA/p117-cj7-cimA(M)-leuBCD | 0.15 |

As disclosed in Table 2 above, a wild-type strain ATCC13032 having ilvA gene produced 0.02 g/L of L-isoleucine, and ATCC13032/p117-IBGC, a transformed strain having the genes in the biosynthesis pathway of L-isoleucine concurrently introduced, produced 0.70 g/L of L-isoleucine, showing that L-isoleucine productivity increased by 3400% compared with the wild-type strain.

Further, transformed strains ATCC13032/p117-cj7-cimA(M) and ATCC13032/p117-cj7-cimA(M)-leuBCD produced 0.83 g/L and 1.25 g/L of L-isoleucine, respectively, showing that L-isoleucine productivity increased by 4050% and 6150%, respectively, compared with the wild-type strain.

From the comparison, the L-isoleucine productivity of ATCC13032/p117-cj7-cimA(M), a strain transformed to introduce a cimA gene into the existing biosynthesis pathway of L-isoleucine, was confirmed as being superior to that of ATCC13032/p117-IBGC, a strain transformed to enhance the biosynthesis pathway of L-isoleucine itself. Further, the L-isoleucine productivity of ATCC13032/p117-cj7-cimA(M)-leuBCD, a transformed strain further enhanced with leuBCD gene, was confirmed as being further capable.

Meanwhile, the ilvA gene-deleted ATCC13032ΔilvA strain lost the L-isoleucine productivity, whereas the ATCC13032ΔilvA/p117-cj7-cimA(M) strain having cimA gene introduced produced 0.08 g/L of L-isoleucine, and the ATCC13032ΔilvA/p117-cj7-cimA(M)-leuBCD strain having cimA gene introduced and a leuBCD gene enhanced produced 0.15 g/L thereof.

The above results show that producing L-isoleucine is possible using a novel biosynthesis pathway of L-isoleucine by the introduction of cimA gene, not the existing biosynthesis pathway of L-isoleucine using L-threonine as a precursor.

### Example 3: Preparation of a Transformed Recombinant Strain and Comparison of L-Isoleucine Productivity

<3-1> Preparation of a Recombinant Strain Using a Wild-Type Microorganism of the Genus Corynebacterium

To provide acetyl-CoA used as a precursor of cimA gene, acetate-auxotrophic strain of wild-type Corynebacterium glutamicum ATCC13032 in which aceE gene encoding pyruvate dehydrogenase is deleted was prepared, and was labeled as “ATCC13032ΔaceE” (Schreiner et al., J Bacteriol. 187: 6005-18, 2005).

Pyruvate dehydrogenase derived from Corynebacterium glutamicum ATCC13032 has the amino acid sequence of SEQ ID NO: 25, and aceE gene encoding the same has the nucleotide sequence of SEQ ID NO: 26.

Recombinant vector p117-cj7-cimA(M) and mutant library of p117-cj7-cimA(M)m prepared in Example 1 above were introduced into the ATCC13032ΔaceE strain prepared above by electroporation. The constructed strains were labeled as “ATCC13032ΔaceE/p117-cj7-cimA(M)” and “ATCC13032ΔaceE/p117-cj7-cimA(M)m mutant library”, respectively.

The ATCC13032ΔaceE, ATCC13032ΔaceE/p117-cj7-cimA(M), and ATCC13032ΔaceE/p117-cj7-cimA(M)m mutant library were plated on a solid medium containing 25 μg/mL of kanamycin to select a single colony, and titer evaluation on L-isoleucine productivity was performed using the selected strains.

Specifically, the strains were inoculated into a 250 mL flask containing 25 mL of a titer medium containing acetate in the form of ammonium acetate having the composition shown in Table 3 below, and then were cultured in an incubator at 32° C. and 200 rpm for 30 hours. As a result, seven types of colonies having increased L-isoleucine concentration were selected, and the list and L-isoleucine concentrations thereof are shown in Table 4 below.

TABLE 3

| Composition | Concentration (per liter) | |
|---|---|---|
| Glucose | 60 | g |
| C₂H₇NO₂ | 5 | g |
| KH₂PO₄ | 1.1 | g |
| (NH₄)₂SO₄ | 20 | g |
| MgSO₄•7H₂O | 1.2 | g |
| FeSO₄•7H₂O | 90 | mg |
| MnSO₄•4H₂O | 90 | mg |
| Thiamine-HCl | 4.5 | mg |
| d-Biotin | 0.9 | mg |
| HSM | 20 | g |
| Calcium carbonate | 30 | g |

| | |
|---|---|
| pH | 7.0 |

TABLE 4

| Strain | L-isoleucine (g/L) |
|---|---|
| ATCC13032ΔaceE | 0.05 |
| ATCC13032ΔaceE/p117-cj7-cimA(M) | 0.84 |
| ATCC13032ΔaceE/p117-cj7-cimA(M)m1 | 1.02 |
| ATCC13032ΔaceE/p117-cj7-cimA(M)m2 | 1.35 |
| ATCC13032ΔaceE/p117-cj7-cimA(M)m3 | 2.23 |
| ATCC13032ΔaceE/p117-cj7-cimA(M)m4 | 1.24 |
| ATCC13032ΔaceE/p117-cj7-cimA(M)m5 | 0.96 |
| ATCC13032ΔaceE/p117-cj7-cimA(M)m6 | 1.73 |
| ATCC13032ΔaceE/p117-cj7-cimA(M)m7 | 0.97 |

As a result, as disclosed in Table 4 above, the ATCC13032ΔaceE strain as a parent strain produced 0.05 g/L of L-isoleucine, and transformed strains produced 0.84 g/L to 2.23 g/L of L-isoleucine, showing that the L-isoleucine productivity increased by 1580% to 4360%, respectively, compared with the parent strain. In particular, transformed strain ATCC13032ΔaceE/p117-cj7-cimA(M)m3 recorded the highest L-isoleucine productivity among the strains compared with the parent strain ATCC13032ΔaceE.

To identify the mutation site of the mutants of cimA(M) gene selected by the increased productivity of L-isoleucine shown in Table 4, and the substituted amino acids of each mutation, sequencing was performed.

As a result, as the CimA(M)m mutant library,

CimA(M)m1 has the amino acid sequence of SEQ ID NO: 3, and may be encoded by a polynucleotide having the nucleotide sequence of SEQ ID NO: 4;

CimA(M)m2 has the amino acid sequence of SEQ ID NO: 5, and may be encoded by a polynucleotide having the nucleotide sequence of SEQ ID NO: 6;

CimA(M)m3 has the amino acid sequence of SEQ ID NO: 7, and may be encoded by a polynucleotide having the nucleotide sequence of SEQ ID NO: 8;

CimA(M)m4 has the amino acid sequence of SEQ ID NO: 9, and may be encoded by a polynucleotide having the nucleotide sequence of SEQ ID NO: 10;

CimA(M)m5 has the amino acid sequence of SEQ ID NO: 11, and may be encoded by a polynucleotide having the nucleotide sequence of SEQ ID NO: 12;

CimA(M)m6 has the amino acid sequence of SEQ ID NO: 13, and may be encoded by a polynucleotide having the nucleotide sequence of SEQ ID NO: 14; and

CimA(M)m7 has the amino acid sequence of SEQ ID NO: 15, and may be encoded by a polynucleotide having the nucleotide sequence of SEQ ID NO: 16.

<3-2> Preparation of a Recombinant Strain Using p117-Cj7-cimA(M)m3 Having the Highest Activity of the Selected p117-cimA(M)m Mutant Library

The p117-cj7-cimA(M)m3 vector identified as having the highest L-isoleucine productivity in Example <3-1> above was treated with XbaI, and was ligated with a DNA fragment recovered by treating a leuBCD fragment recovered in Example <1-3> above with the same restriction enzyme.

E. coli DH5a cells were heat-shock transformed with the constructed vector, then plated on a 25 μg/mL kanamycin-containing LB solid medium, and cultured overnight at 37° C. One platinum loop of the cultured colony was inoculated into 3 mL of a 25 μg/mL kanamycin-containing LB liquid medium and cultured overnight, and then plasmid DNA was recovered using a plasmid miniprep kit.

The construction of the recombinant vector was confirmed by treatment with restriction enzyme XbaI, and the clone was identified by performing PCR using the primer pair of SEQ ID NOS: 29 and 30 under the following conditions: 30 cycles, each consisting of denaturation at 95° C. for 30 sec, annealing at 56° C. for 30 sec, and elongation at 72° C. for 180 sec. The recovered recombinant vector was labeled as “p117-cj7-cimA(M)m3-leuBCD”.

Each of 17-cj7-cimA(M) of Example <1-2>, p117-cj7-cimA(M)-leuBCD of Example <1-4>, p117-cj7-cimA(M)m3 of Example <3-1>, and p117-cj7-cimA(M)m3-leuBCD of Example <3-2> above was introduced into the ATCC13032ΔaceE strain used in Example <3-1> by electroporation, and was plated on a solid medium containing 25 μg/mL of kanamycin to select a single colony.

The selected strains were inoculated into a 250 mL flask containing 25 mL of a titer medium containing glucose having the composition shown in Table 3 above, and then were cultured in an incubator at 32° C. and 200 rpm for 30 hours to examine the L-isoleucine productivity. The results are shown in Table 5 below.

TABLE 5

| Strain | L-isoleucine (g/L) |
|---|---|
| ATCC13032ΔaceE | 0.04 |
| ATCC13032ΔaceE/p117-cj7-cimA(M) | 0.98 |
| ATCC13032ΔaceE/p117-cj7-cimA(M)-leuBCD | 2.09 |
| ATCC13032ΔaceE/p117-cj7-cimA(M)m3 | 2.22 |
| ATCC13032ΔaceE/p117-cj7-cimA(M)m3-leuBCD | 3.76 |

As disclosed in Table 5 above, while the ATCC13032ΔaceE strain as a parent strain produced 0.04 g/L of L-isoleucine, transformed strain ATCC13032ΔaceE/p117-cj7-cimA(M) produced 0.98 g/L of L-isoleucine and ATCC13032ΔaceE/p117-cj7-cimA(M)-leuBCD produced 2.09 g/L of L-isoleucine, showing increases of 0.94 g/L and 2.05 g/L compared with the L-isoleucine productivity of the parent strain.

Further, transformed strains ATCC13032ΔaceE/p117-cj7-cimA(M)m3 and ATCC13032ΔaceE/p117-cj7-cimA(M)m3-leuBCD including the cimA(M)m3 mutant gene recovered in Example <3-1> produced 2.22 g/L and 3.76 g/L of L-isoleucine, respectively, which increased by 5450% and 9300% of the production compared with the parent strain. Accordingly, this result proves that the cimA(M)m3 mutant is more effective than a wild-type strain thereof in the increase of the L-isoleucine productivity.

Among the transformed strains recovered in the above Examples, ATCC13032ΔaceE/p117-cj7-cimA(M)m3 was labeled as “Corynebacterium glutamicum CA10-1002”, deposited to the Korean Culture Center of Microorganisms (KCCM) under the Budapest Treaty on Feb. 27, 2015, and was provided with Accession No. KCCM11672P.

<3-3> Preparation of a Recombinant Strain Using L-Isoleucine-Producing Strain KCCM11248P

Each of p117-cj7-cimA(M), p117-cj7-cimA(M)-leuBCD, p117-cj7-cimA(M)m3, and p117-cj7-cimA(M)m3-leuBCD, the recombinant vectors used in Examples <3-1> and <3-2>, was introduced into Corynebacterium glutamicum KCCM11248P, an L-isoleucine-producing strain (Korean Patent No. 10-1335789), and was plated on a solid medium containing 25 μg/mL of kanamycin to select a single colony.

Each of the selected strains was labeled as KCCM11248P/p117-cj7-cimA(M), KCCM11248P/p117-cj7-cimA(M)-leuBCD, KCCM11248P/p117-cj7-cimA(M)m3, and KCCM11248P/p117-cj7-cimA(M)m3-leuBCD.

The L-isoleucine productivity of the strains was examined using L-isoleucine titer media having the composition shown in Table 6 below and culturing the strains in flasks as described below.

TABLE 6

| Composition | Concentration (per liter) | |
|---|---|---|
| Glucose | 45 | g |
| BM | 10 | g |
| KH₂PO₄ | 1.1 | g |
| (NH₄)₂SO₄ | 12.5 | g |
| MgSO₄•7H₂O | 1.2 | g |
| FeSO₄•7H₂O | 180 | mg |
| MnSO₄•4H₂O | 180 | mg |
| ZnSO₄•5H₂O | 0.9 | mg |
| CuSO₄•5H₂O | 0.9 | mg |
| Thiamine-HCl | 9 | mg |
| d-Biotin | 1.8 | mg |
| Ca-Pantothenate | 9 | mg |
| NCA | 60 | mg |
| HSM | 20 | g |
| Calcium carbonate | 30 | g |

| | |
|---|---|
| pH | 7.0 |

Specifically, parent strain KCCM11248P and transformed strains KCCM11248P/p117-cj7-cimA(M), KCCM11248P/p117-cj7-cimA(M)-leuBCD, KCCM11248P/p117-cj7-cimA(M)m3, and KCCM11248P/p117-cj7-cimA(M)m3-leuBCD were inoculated into 250 mL flasks containing 25 mL of titer media containing glucose having the composition shown in Table 6, and then were cultured in an incubator at 32° C. and 200 rpm for 60 hours to examine the L-isoleucine productivity. The results are shown in Table 7 below.

TABLE 7

| Strain | L-isoleucine (g/L) |
|---|---|
| KCCM11248P | 3.33 |
| KCCM11248P/p117-cj7-cimA(M) | 4.04 |
| KCCM11248P/p117-cj7-cimA(M)-leuBCD | 4.82 |
| KCCM11248P/p117-cj7-cimA(M)m3 | 4.71 |
| KCCM11248P/p117-cj7-cimA(M)m3-leuBCD | 11.21 |

As disclosed in Table 7 above, while the parent strain KCCM11248P produced 3.33 g/L of L-isoleucine, transformed strains KCCM11248P/p117-cj7-cimA(M) produced 4.04 g/L of L-isoleucine, and KCCM11248P/p117-cj7-cimA(M)-leuBCD produced 4.82 g/L of L-isoleucine, showing increases of 0.71 g/L and 1.49 g/L compared with the L-isoleucine productivity of the parent strain.

Meanwhile, the transformed strains KCCM11248P/p117-cj7-cimA(M)m3 and KCCM11248P/p117-cj7-cimA(M)m3-leuBCD produced 4.71 g/L and 11.21 g/L of L-isoleucine, respectively, which increased in production by 41.4% and 236.6% compared with the parent strain.

As shown in the result of Example <3-2> using Corynebacterium glutamicum ATCC13032, this result further proves that the cimA(M)m3 mutant is more effective than a wild-type strain thereof in the increase of the L-isoleucine productivity.

From the foregoing, a skilled person in the art to which the present disclosure pertains will be able to understand that the present disclosure may be embodied in other specific forms without modifying the technical concepts or essential characteristics of the present disclosure. In this regard, the exemplary embodiments disclosed herein are only for illustrative purposes and should not be construed as limiting the scope of the present disclosure. On the contrary, the present disclosure is intended to cover not only the exemplary embodiments but also various alternatives, modifications, equivalents, and other embodiments that may be included within the spirit and scope of the present disclosure as defined by the appended claims.

1. A method for producing L-isoleucine, comprising:
culturing a microorganism of the genus Corynebacterium comprising a protein having an activity of citramalate synthase in a medium; and
recovering L-isoleucine from the microorganism or medium.

2. The method of claim 1, wherein the citramalate synthase is derived from a microorganism of the genus Methanocaldococcus.

3. The method of claim 1, wherein the citramalate synthase has an amino acid sequence selected from the group consisting of SEQ ID NOS: 1, 3, 5, 7, 9, 11, 13, and 15.

4. The method of claim 1, wherein the microorganism of the genus Corynebacterium further comprises enhanced activities of 3-isopropylmalate dehydrogenase and 3-isopropylmalate dehydratase.

5. The method of claim 1, wherein the microorganism of the genus Corynebacterium further comprises inactivated pyruvate dehydrogenase.

6. The method of claim 1, wherein acetate is further provided in culturing the microorganism of the genus Corynebacterium.

7. The method of claim 1, wherein the microorganism of the genus Corynebacterium is Corynebacterium glutamicum.

8. A microorganism of the genus Corynebacterium having L-isoleucine producing ability, wherein the microorganism comprises a protein having an activity of citramalate synthase.

9. The microorganism of claim 8, wherein the citramalate synthase has an amino acid sequence selected from the group consisting of SEQ ID NOS: 1, 3, 5, 7, 9, 11, 13, and 15.

10. The microorganism of claim 8, wherein the microorganism further comprises enhanced activities of 3-isopropylmalate dehydrogenase and 3-isopropylmalate dehydratase.

11. The microorganism of claim 8, wherein the microorganism further comprises inactivated pyruvate dehydrogenase.

12. The microorganism of claim 8, wherein the microorganism is Corynebacterium glutamicum.


SEQUENCE LISTING (52 sequences)

SEQ ID NO: 1 - protein, 491 aa, Methanocaldococcus jannaschii
  cimA
MMVRIFDTTLRDGEQTPGVSLTPNDKLEIAKKLDELGVDVIEAGSAITSKGEREGIKLITKEGLNAEICSFVRALPVDIDAALECDVDSVHLVVPTSPIHMKYKLRKTEDEVLETALKAVEYAKEHGLIVELSAEDATRSDVNFLIKLFNEGEKVGADRVCVCDTVGVLTPQKSQELFKKITENVNLPVSVHCHNDFGMATANTCSAVLGGAVQCHVTVNGIGERAGNASLEEVVAALKILYGYDTKIKMEKLYEVSRIVSRLMKLPVPPNKAIVGDNAFAHEAGIHVDGLIKNTETYEPIKPEMVGNRRRIILGKHSGRKALKYKLDLMGINVSDEQLNKIYERVKEFGDLGKYISDADLLAIVREVTGKLVEEKIKLDELTVVSGNKITPIASVKLHYKGEDITLIETAYGVGPVDAAINAVRKAISGVADIKLVEYRVEAIGGGTDALIEVVVKLRKGTEIVEVRKSDADIIRASVDAVMEGINMLLN

SEQ ID NO: 2 - DNA, 1476 bp, Methanocaldococcus jannaschii
  cimA
atgatggtaaggatatttgatacaacacttagagatggagagcaaacaccaggagtttctttaacaccaaatgataagttagagatagcaaaaaaattggatgagcttggagttgatgttatagaggcaggttcagctataacttcaaaaggagagagagaaggaataaaattaataacaaaagaaggtttaaatgcagaaatctgctcatttgttagagctttacctgtagatattgatgctgccttagaatgtgatgtagatagtgtccatttagtagtgccaacatctccaatacacatgaaatataagcttagaaaaacagaagatgaggttttagagacagctttaaaggctgtagagtatgctaaagaacatggattgattgttgagttatctgcagaggatgcaacaagaagtgatgtaaatttcttaataaaactatttaatgaaggggaaaaggttggagcagacagagtttgtgtttgtgacacagtaggagttttaactccacaaaagagtcaggaattatttaaaaaaataactgaaaatgttaatttaccggtctcagttcattgccacaacgactttggaatggctactgctaatacttgctcagcagttttaggtggagctgttcagtgccacgtaacagttaatggtattggagagagagcaggaaatgcctcattggaagaggttgttgctgctttaaaaatactctatggctatgatactaagataaagatggaaaagttatatgaggtttcaagaattgtctcaagattgatgaaacttcctgttccaccaaataaagcaattgttggggacaatgcatttgctcatgaagcaggaatacatgttgatggattaataaaaaatactgaaacctatgagccaataaaaccagaaatggttgggaatagaagaagaattattttgggtaagcattctggtagaaaagctttaaaatacaaacttgatttgatgggcataaacgttagtgatgagcaattaaataaaatatatgaaagagttaaagaatttggggatttgggtaaatacatttcagacgctgatttgttggctatagttagagaagttactggaaaattggtagaagagaaaatcaaattagatgaattaactgttgtatctggaaataaaataacaccaattgcatctgttaaactccattataaaggagaagatataactttaatagaaactgcttatggtgttggaccggtagatgcagcaataaatgctgtgagaaaggcaataagtggagttgcagatattaagttggtagagtatagagttgaagcaattggtggaggaactgatgcgttaatagaggttgttgttaaattaagaaaaggaactgaaattgttgaagttagaaaatcagacgctgatataataagggcttctgtagatgctgtaatggaaggaatcaatatgttattgaattaa

SEQ ID NO: 3 - protein, 491 aa, Artificial Sequence
  Description of Artificial Sequence Synthetic CimA(M)m1 polypeptide
MMVRIFDTTLRDGEQTPGVSLTPNDKLEIAKKLDELGVDVIEAGSAVTSKGEREGIKLITKEGLNAEICSFVRALPVDIDAALECDVDSVHLVVPTSPIHMKYKLRKTEDEVLETALKAVEYAKEHGLIVELSAEDATRSDVNFLIKLFNEGEKVGADRVCVCDTVGVLTPQKSQELFKKITKNVNLPVSVHCHNDFGMATANACSAVLGGAVQCHVTVNGIGERAGNASLEEVVAALKILYGYDTKIKMEKLYEVSRIVSRLMKLPVPPNKAIVGDNAFAHEAGIHVDGLIKNTETYEPIKPEMVGNRRRIILGKHSGRKALKYKLDLMGINVSDEQLNKIYERVKEFGDLGKYISDADLLAIVREVTGKLVEEKIKLDELTVVSGNKITPIASVKLHYKGEDIRLIETAYGVGPVDAAINAVRKAISGVADIKLVEYRVEAIGGGTDALIEVVVKLRKGTEIVEVRKSDADIIRASVDAVMEGINMLLN

SEQ ID NO: 4 - DNA, 1476 bp, Artificial Sequence
  Description of Artificial Sequence Synthetic cimA(M)m1 polynucleotide
atgatggtaaggatatttgatacaacacttagagatggagagcaaacaccaggagtttctttaacaccaaatgataagttagagatagcaaaaaaattggatgagcttggagttgatgttatagaggcaggttcagctgtaacttcaaaaggagagagagaaggaataaaattaataacaaaagaaggtttaaatgcagaaatctgctcatttgttagagctttacctgtagatattgatgctgccttagaatgtgatgtagatagtgtccatttagtagtgccaacatctccaatacacatgaaatataagcttagaaaaacagaagatgaggttttagagacagctttaaaggctgtagagtatgctaaagaacatggattgattgttgagttatctgcagaggatgcaacaagaagtgatgtaaatttcttaataaaactatttaatgaaggggaaaaggttggagcagacagagtttgtgtttgtgacacagtaggagttttaactccacaaaagagtcaggaattatttaaaaaaataactaaaaatgttaatttaccggtctcagttcattgccacaacgactttggaatggctactgctaatgcttgctcagcagttttaggtggagctgttcagtgccacgtaacagttaatggtattggagagagagcaggaaatgcctcattggaagaggttgttgctgctttaaaaatactctatggctatgatactaagataaagatggaaaagttatatgaggtttcaagaattgtctcaagattgatgaaacttcctgttccaccaaataaagcaattgttggggacaatgcatttgctcatgaagcaggaatacatgttgatggattaataaaaaatactgaaacctatgagccaataaaaccagaaatggttgggaatagaagaagaattattttgggtaagcattctggtagaaaagctttaaaatacaaacttgatttgatgggcataaacgttagtgatgagcaattaaataaaatatatgaaagagttaaagaatttggggatttgggtaaatacatttcagacgctgatttgttggctatagttagagaagttactggaaaattggtagaagagaaaatcaaattagatgaattaactgttgtatctggaaataaaataacaccaattgcatctgttaaactccattataaaggagaagatataaggttaatagaaactgcttatggtgttggaccggtagatgcagcaataaatgctgtgagaaaggcaataagtggagttgcagatattaagttggtagagtatagagttgaagcaattggtggaggaactgatgcgttaatagaggttgttgttaaattaagaaaaggaactgaaattgttgaagttagaaaatcagacgctgatataataagggcttctgtagatgctgtaatggaaggaatcaatatgttattgaattaa

SEQ ID NO: 5 - protein, 491 aa, Artificial Sequence
  Description of Artificial Sequence Synthetic CimA(M)m2 polypeptide
MMVRIFDTTLRDGEQTPGVSLTPNDKLEIAKKLDELGVDVIEAGSAITSKGEGEGIKLITKEGLNAEICSFVRALPVDIDAALECDVDSVHLVVPTSPIHMKYKLRKTEDEVVETALKAVEYAKEQGLIVELSAEDATRSDVNFLIKLFNEGEKVGADRVCVCDTVGVLTPQKSQELFKKITENVNLPVSVHCHNDFGMATANACSAVLGGAVQCHVTVNGIGERAGNASLEEVVAALKILYGYDTKIKMEKLYEVSRIVSRLMELPVPPNKAIVGDNAFAHEAGIHVDGLIKNTETYEPIKPEMVGNRRRIILGKHSGRKALKYKLDLMGINVSDEQLNKIYERVKEFGDLGKYISDADLLAIVREVTGKLVEEKIKLDELTVVSGNKITPIASVKLHYKGEDITLIETAYGVGPVDAAINAVRKAISGVADIKLVEYRVEAIGGGTDALIEVVVKLRKGTEIVEVRKSDADIIEASVDAVMEGINMLLN

SEQ ID NO: 6 - DNA, 1476 bp, Artificial Sequence
  Description of Artificial Sequence Synthetic cimA(M)m2 polynucleotide
atgatggtaaggatatttgatacaacacttagagatggagagcaaacaccaggagtttctttaacaccaaatgataagttagagatagcaaaaaaattggatgagcttggagttgatgttatagaggcaggttcagctataacttcaaaaggagagggagaaggaataaaattaataacaaaagaaggtttaaatgcagaaatctgctcatttgttagagctttacctgtagatattgatgctgccttagaatgtgatgtagatagtgtccatttagtagtgccaacatctccaatacacatgaaatataagcttagaaaaacagaagatgaggttgtagagacagctttaaaggctgtagagtatgctaaagaacaaggattgattgttgagttatctgcagaggatgcaacaagaagtgatgtaaatttcttaataaaactatttaatgaaggggaaaaggttggagcagacagagtttgtgtttgtgacacagtaggagttttaactccacaaaagagtcaggaattatttaaaaaaataactgaaaatgttaatttaccggtctcagttcattgccacaacgactttggaatggctactgctaatgcttgctcagcagttttaggtggagctgttcagtgccacgtaacagttaatggtattggagagagagcaggaaatgcctcattggaagaggttgttgctgctttaaaaatactctatggctatgatactaagataaagatggaaaagttatatgaggtttcaagaattgtctcaagattgatggagcttcctgttccaccaaataaagcaattgttggggacaatgcatttgctcatgaagcaggaatacatgttgatggattaataaaaaatactgaaacctatgagccaataaaaccagaaatggttgggaatagaagaagaattattttgggtaagcattctggtagaaaagctttaaaatacaaacttgatttgatgggcataaacgttagtgatgagcaattaaataaaatatatgaaagagttaaagaatttggggatttgggtaaatacatttcagacgctgatttgttggctatagttagagaagttactggaaaattggtagaagagaaaatcaaattagatgaattaactgttgtatctggaaataaaataacaccaattgcatctgttaaactccattataaaggagaagatataactttaatagaaactgcttatggtgttggaccggtagatgcagcaataaatgctgtgagaaaggcaataagtggagttgcagatattaagttggtagagtatagagttgaagcaattggtggaggaactgatgcgttaatagaggttgttgttaaattaagaaaaggaactgaaattgttgaagttagaaaatcagacgctgatataatagaggcttctgtagatgctgtaatggaaggaatcaatatgttattgaattaa

SEQ ID NO: 7 - protein, 372 aa, Artificial Sequence
  Description of Artificial Sequence Synthetic CimA(M)m3 polypeptide
MMVRIFDTTLRDGEQTPGVSLTPNDKLEIAKKLDELGVDVIEAGSAVTSKGEREGIKLITKEGLNAEICSFVRALPVDIDAALECDVDSVHLVVPTSPIHMKYKLRKTEDEVLVTALKAVEYAKEQGLIVELSAEDATRSDVNFLIKLFNEGEKVGADRVCVCDTVGVLTPQKSQELFKKITENVNLPVSVHCHNDFGMATANACSAVLGGAVQCHVTVNGIGERAGNASLEEVVAASKILYGYDTKIKMEKLYEVSRIVSRLMKLPVPPNKAIVGDNAFAHEAGIHVDGLIKNTETYEPIKPEMVGNRRRIILGKHSGRKALKYKLDLMGINVSDEQLNKIYERVKEFGDLGKYISDADLLAIVREVTGKL

SEQ ID NO: 8 - DNA, 1119 bp, Artificial Sequence
  Description of Artificial Sequence Synthetic cimA(M)m3 polynucleotide
atgatggtaaggatattcgataccacacttcgcgacggagagcagacaccaggagtttctttaacaccaaacgataagctggagatagctaaaaagttggatgaacttggggttgatgttatagaggccggtagcgctgtcactagcaagggagagcgcgaaggaataaaactcattacaaaagaaggtcttaacgcagaaatctgcagtttcgtgcgtgcactccctgtagacatagacgctgccttagaatgtgatgtagatagtgtgcacctcgtggtgcccacatctccaatacacatgaagtacaaacttcgtaaaacggaggatgaggttctcgtgaccgcacttaaggctgtggaatacgctaaagagcagggcttgatcgttgagttatcagcggaggatgcgacaagatccgatgttaatttcttaataaaactcttcaatgaaggggaaaaggttggagcagaccgtgtttgtgtttgcgacaccgtaggagttttaacgccacaaaagtcgcaggaactgtttaagaaaataaccgagaatgtgaatttaccggtctcggttcattgccacaacgactttggaatggctactgctaatgcttgctccgcagtgctcggcggagctgttcagtgccacgtgacagttaacggtattggcgagcgcgcagggaatgcctccttagaagaggtcgttgccgcgtcaaaaatcctctacggctatgatactaaaataaagatggaaaagttatacgaggtttcacgcattgtctcccgcttgatgaaacttcctgttcccccgaacaaagcgattgttggcgacaatgcgttcgctcacgaagcaggtatccatgtcgatggattaattaaaaatactgaaacctatgagccaatcaaaccagaaatggtgggcaaccgccggcgtatcattctcggtaagcactctggtcggaaagcgttaaagtacaagcttgatctaatgggcataaacgtgtctgatgagcaattaaacaaaatttatgaacgcgttaaagaattcggggacctaggtaagtacatttcagatgctgatttgctcgctatagttcgcgaagtcacgggaaagttgtaa

SEQ ID NO: 9 - protein, 372 aa, Artificial Sequence
  Description of Artificial Sequence Synthetic CimA(M)m4 polypeptide
MMVRIFDTTLRDGEQTPGVSLTPNDKLEIAKKLDELGVDVIEAGSAVTSKGEREGIKLITKEGLNAEICSFVRALPVDIDAALECDVDSVHLVVPTSPIHMKYKLRKTEDEVLETALKAVEYAKEQGLIVELSAEDATRSDVNFLIKLFNEGEKVGADRVCVCDTVGVLTPQKSQELFKKITENVNLPVSVHCHNDFGMATANTCSAVLGGAVQCHVTVNGIGERAGNASLEEVVAALKILYGYDTKIKMEKSYEVSRIVSRLMKLPVPPNKAIVGDNAFAHEAGIHVDGLIKNTETYEPIKPEMVGNRRRIILGKHSGRKALKYKLDLMGINVSDEQLNKIYERVKEFGDLGKYISDADLLAIVREVTGKL

SEQ ID NO: 10 - DNA, 1119 bp, Artificial Sequence
  Description of Artificial Sequence Synthetic cimA(M)m4 polynucleotide
atgatggtaaggatatttgatacaacacttagagatggagagcaaacaccaggagtttctttaacaccaaatgataagttagagatagcaaaaaaattggatgagcttggagttgatgttatagaggcaggttcagctgtaacttcaaaaggagagagagaaggaataaaattaataacaaaagaaggtttaaatgcagaaatctgctcatttgttagagctttacctgtagatattgatgctgccttagaatgtgatgtagatagtgtccatttagtagtgccaacatctccaatacacatgaaatataagcttagaaaaacagaagatgaggttttagagacagctttaaaggctgtagagtatgctaaagaacaaggattgattgttgagttatctgcagaggatgcaacaagaagtgatgtaaatttcttaataaaactatttaatgaaggggaaaaggttggagcagacagagtttgtgtttgtgacacagtaggagttttaactccacaaaagagtcaggaattatttaaaaaaataactgaaaatgttaatttaccggtctcagttcattgccacaacgactttggaatggctactgctaatacttgctcagcagttttaggtggagctgttcagtgccacgtaacagttaatggtattggagagagagcaggaaatgcctcattggaagaggttgttgctgctttaaaaatactctatggctatgatactaagataaagatggaaaagtcatatgaggtttcaagaattgtctcaagattgatgaaacttcctgttccaccaaataaagcaattgttggggacaatgcatttgctcatgaagcaggaatacatgttgatggattaataaaaaatactgaaacctatgagccaataaaaccagaaatggttgggaatagaagaagaattattttgggtaagcattctggtagaaaagctttaaaatacaaacttgatttgatgggcataaacgttagtgatgagcaattaaataaaatatatgaaagagttaaagaatttggggatttgggtaaatacatttcagacgctgatttgttggctatagttagagaagttactggaaaattgtga

SEQ ID NO: 11 - protein, 491 aa, Artificial Sequence
  Description of Artificial Sequence Synthetic CimA(M)m5 polypeptide
MMVRIFDTTLRDGEQTPGVSLTPNDKLEIAKKLDELGVDVIEAGSAITSKGEREGIKLITKEGLNAEICSFVRALPVDIDAALECDVDSVHLVVPTSPIHMKYKLRKTEDEVLETALKAVEYAKEHGLIVELSAEDATRSDVNFLIKLFNEGEKVGADRVCVCDTVGVLTPQKSQELFKKITENVNLPVSVHCHNDFGMATANACSAVLGGAVQCHVTVNGIGERAGNASLEEVVAASKILYGYDTKIKMEKLYEVSRIVSRLMKLPVPPNKAIVGDNAFAHEAGIHVDGLIKNTETYEPIKPEMVGNRRRIILGKHSGRKALKYKLDLMGINVSDEQLNKIYERVKEFGDLGKYISDADLLAIVREVTGKLVEEKIKLDELTVVSGNKITPIASVKLHYKGEDITLIETAYGVGPVDAAINAVRKAISGVADIKLVEYRAEAIGGGTDALIEVVVKLRKGTEIVEVRKSDADIIRASVDAVMEGINMLLN

SEQ ID NO: 12 - DNA, 1476 bp, Artificial Sequence
  Description of Artificial Sequence Synthetic cimA(M)m5 polynucleotide
atgatggtaaggatatttgatacaacacttagagatggagagcaaacaccaggagtttctttaacaccaaatgataagttagagatagcaaaaaaattggatgagcttggagttgatgttatagaggcaggttcagctataacttcaaaaggagagagagaaggaataaaattaataacaaaagaaggtttaaatgcagaaatctgctcatttgttagagctttacctgtagatattgatgctgccttagaatgtgatgtagatagtgtccatttagtagtgccaacatctccaatacacatgaaatataagcttagaaaaacagaagatgaggttttagagacagctttaaaggctgtagagtatgctaaagaacatggattgattgttgagttatctgcagaggatgcaacaagaagtgatgtaaatttcttaataaaactatttaatgaaggggaaaaggttggagcagacagagtttgtgtttgtgacacagtaggagttttaactccacaaaagagtcaggaattatttaaaaaaataactgaaaatgttaatttaccggtctcagttcattgccacaacgactttggaatggctactgctaatgcttgctcagcagttttaggtggagctgttcagtgccacgtaacagttaatggtattggagagagagcaggaaatgcctcattggaagaggttgttgctgcttcaaaaatactctatggctatgatactaagataaagatggaaaagttatatgaggtttcaagaattgtctcaagattgatgaaacttcctgttccaccaaataaagcaattgttggggacaatgcatttgctcatgaagcaggaatacatgttgatggattaataaaaaatactgaaacctatgagccaataaaaccagaaatggttgggaatagaagaagaattattttgggtaagcattctggtagaaaagctttaaaatacaaacttgatttgatgggcataaacgttagtgatgagcaattaaataaaatatatgaaagagttaaagaatttggggatttgggtaaatacatttcagacgctgatttgttggctatagttagagaagttactggaaaattggtagaagagaaaatcaaattagatgaattaactgttgtatctggaaataaaataacaccaattgcatctgttaaactccattataaaggagaagatataactttaatagaaactgcttatggtgttggaccggtagatgcagcaataaatgctgtgagaaaggcaataagtggagttgcagatattaagttggtagagtatagagccgaagcaattggtggaggaactgatgcgttaatagaggttgttgttaaattaagaaaaggaactgaaattgttgaagttagaaaatcagacgctgatataataagggcttctgtagatgctgtaatggaaggaatcaatatgttattgaattaa

SEQ ID NO: 13 - protein, 491 aa, Artificial Sequence
  Description of Artificial Sequence Synthetic CimA(M)m6 polypeptide
MMVRIFDTTLRDGEQTPGVSLTPNDKLEIAKKLDELGVDVIEAGSAVTSKGEREGIKLITKEGLNAEICSFVRALPVDIDAALECDVDSVHLVVPTSPIHMKYKLRKTEDEVLETALKAVEYAKEQGLIVELSAEDATRSDVNFLIKLFNEGEKVGADRVCVCDTVGVLTPQKSQELFKKITENVNLPVSVHCHNDFGMATANACSAVLGGAVQCHVTVNGIGERAGNASLEEVVAALKILYGYDTKIKMEKLYEVSRIVSRLMKLPVPPNKAIVGDNAFAHEAGIHVDGLIKNTETYEPIKPEMVGNRRRIILGKHSGRKALKYKLDLMGINVSKEQLNKIYERVKEFGDLGKYISDADLLAIVREVTGKLVEEKIKLDELTVVSGNKITPIASVKLHYKGEDITLIETAYGVGPVDAAINAVRKAISGVADINLVEYRVEAIGGGTDALIEVVVKLRKGTEIVEVRKSDADIIRASVDAVMEGINMLLN

SEQ ID NO: 14 - DNA, 1476 bp, Artificial Sequence
  Description of Artificial Sequence Synthetic cimA(M)m6 polynucleotide
atgatggtaaggatatttgatacaacacttagagatggagagcaaacaccaggagtttctttaacaccaaatgataagttagagatagcaaaaaaattggatgagcttggagttgatgttatagaggcaggttcagctgtaacttcaaaaggagagagagaaggaataaaattaataacaaaagaaggtttaaatgcagaaatctgctcatttgttagagctttacctgtagatattgatgctgccttagaatgtgatgtagatagtgtccatttagtagtgccaacatctccaatacacatgaaatataagcttagaaaaacagaagatgaggttttagagacagctttaaaggctgtagagtatgctaaagaacaaggattgattgttgagttatctgcagaggatgcaacaagaagtgatgtaaatttcttaataaaactatttaatgaaggggaaaaggttggagcagacagagtttgtgtttgtgacacagtaggagttttaactccacaaaagagtcaggaattatttaaaaaaataactgaaaatgttaatttaccggtctcagttcattgccacaacgactttggaatggctactgctaatgcttgctcagcagttttaggtggagctgttcagtgccacgtaacagttaatggtattggagagagagcaggaaatgcctcattggaagaggttgttgctgctttaaaaatactctatggctatgatactaagataaagatggaaaagttatatgaggtttcaagaattgtctcaagattgatgaaacttcctgttccaccaaataaagcaattgttggggacaatgcatttgctcatgaagcaggaatacatgttgatggattaataaaaaatactgaaacctatgagccaataaaaccagaaatggttgggaatagaagaagaattattttgggtaagcattctggtagaaaagctttaaaatacaaacttgatttgatgggcataaacgttagtaaagagcaattaaataaaatatatgaaagagttaaagaatttggggatttgggtaaatacatttcagacgctgatttgttggctatagttagagaagttactggaaaattggtagaagagaaaatcaaattagatgaattaactgttgtatctggaaataaaataacaccaattgcatctgttaaactccattataaaggagaagatataactttaatagaaactgcttatggtgttggaccggtagatgcagcaataaatgctgtgagaaaggcaataagtggagttgcagatattaacttggtagagtatagagttgaagcaattggtggaggaactgatgcgttaatagaggttgttgttaaattaagaaaaggaactgaaattgttgaagttagaaaatcagacgctgatataataagggcttctgtagatgctgtaatggaaggaatcaatatgttattgaattaa

SEQ ID NO: 15 - protein, 491 aa, Artificial Sequence
  Description of Artificial Sequence Synthetic CimA(M)m7 polypeptide
MMVRIFDTTLRDGEQTPGVSLTPNDKLEIAKKLDELGVDVIEAGSAVTSKGEREGIKLITKEGLNAEICSFVRALPVDIDAALECDVDSVHLVVPTSPIHMKYKLRKTEDEVLMTALKAVEYAKEHGLIVELSAEDATRSDVNFLIKLFNEGEKVGADRVCVCDTVGVLTPQKSQELFKKITENVNLPVSVHCHNDFGMATANTCSAVLGGAVQCHVTVNGIGERAGNASLEEVVAALKILYGYDTKIKQEKLYEVSRIVSRLMKLPVPPNKAIVGDNAFAHEAGIHVDGLIKNTETYEPIKPEMVGNRRRIILGKHSGRKALKYKLDLMGINVSDEQLNKIYERVKEFGDLGKYISDADLLAIVREVTGKLVEEKIKLDELTVVSGNKITPIASVKLHYKGEDITLIETAYGVGPVDAAINAVRKAISGVADIKLVEYRAEAIGGGTDALIEVVVKLRKGTEIVEVRKSDADIIRASVDAVMEGINMLLN

SEQ ID NO: 16 - DNA, 1476 bp, Artificial Sequence
  Description of Artificial Sequence Synthetic cimA(M)m7 polynucleotide
atgatggtaaggatatttgatacaacacttagagatggagagcaaacaccaggagtttctttaacaccaaatgataagttagagatagcaaaaaaattggatgagcttggagttgatgttatagaggcaggttcagctgtaacttcaaaaggagagagagaaggaataaaattaataacaaaagaaggtttaaatgcagaaatctgctcatttgttagagctttacctgtagatattgatgctgccttagaatgtgatgtagatagtgtccatttagtagtgccaacatctccaatacacatgaaatataagcttagaaaaacagaagatgaggttttaatgacagctttaaaggctgtagagtatgctaaagaacatggattgattgttgagttatctgcagaggatgcaacaagaagtgatgtaaatttcttaataaaactatttaatgaaggggaaaaggttggagcagacagagtttgtgtttgtgacacagtaggagttttaactccacaaaagagtcaggaattatttaaaaaaataactgaaaatgttaatttaccggtctcagttcattgccacaacgactttggaatggctactgctaatacttgctcagcagttttaggtggagctgttcagtgccacgtaacagttaatggtattggagagagagcaggaaatgcctcattggaagaggttgttgctgctttaaaaatactctatggctatgatactaagataaagcaggaaaagttatatgaggtttcaagaattgtctcaagattgatgaaacttcctgttccaccaaataaagcaattgttggggacaatgcatttgctcatgaagcaggaatacatgttgatggattaataaaaaatactgaaacctatgagccaataaaaccagaaatggttgggaatagaagaagaattattttgggtaagcattctggtagaaaagctttaaaatacaaacttgatttgatgggcataaacgttagtgatgagcaattaaataaaatatatgaaagagttaaagaatttggggatttgggtaaatacatttcagacgctgatttgttggctatagttagagaagttactggaaaattggtagaagagaaaatcaaattagatgaattaactgttgtatctggaaataaaataacaccaattgcatctgttaaactccattataaaggagaagatataactttaatagaaactgcttatggtgttggaccggtagatgcagcaataaatgctgtgagaaaggcaataagtggagttgcagatattaagttggtagagtatagagctgaagcaattggtggaggaactgatgcgttaatagaggttgttgttaaattaagaaaaggaactgaaattgttgaagttagaaaatcagacgctgatataataagggcttctgtagatgctgtaatggaaggaatcaatatgttattgaattaa

SEQ ID NO: 17 - protein, 340 aa, Corynebacterium glutamicum
  leuB
MKLAVIGGDGIGPEVTAEALKVLNAVRDDIETTDYDLGARRYLKNGELLTDEDLASLREHDAILLGAIGAPGSVPPGILERGLLLKMRFALDHHVNLRPSKLYDGVESPLRNPGKIDFVVVREGTEGAYTGNGGAIRVGTPHEIANETSVNTRYGAERVIRYAFELAQSRRKKLTLVHKTNVLVHGGGLWQRTVDEVAKEYPEVAVDYNHIDAATIYLVTDPSRFDVIVTDNLFGDILTDEAGAVSGGIGLAASGNIDATGTNPSMFEPVHGSAPDIAGQGIADPTAAILSAAMLLRHLGDEDNAVRIETAIAADVAGRDNSQPISTTEVGDRIVKALQS

SEQ ID NO: 18 - DNA, 1023 bp, Corynebacterium glutamicum
  leuB
atgaaacttgctgttattggtggagatggtatcggcccagaggttactgcagaagccctcaaggttctaaacgctgtccgcgacgacatcgagaccaccgattatgaccttggcgcacgccgttacctcaaaaatggcgagctgctcaccgacgaggatctggcatccctgcgcgagcatgacgcgatccttcttggcgctatcggtgcaccaggttccgtccctccaggaattctcgagcgtggtttgctgctgaagatgcgattcgcactggatcaccacgtgaacctgcgcccatccaagctgtacgacggcgtggagtccccactgcgtaacccaggcaagattgatttcgttgtggtccgcgaaggtaccgaaggcgcctacactggcaacggtggagcaatccgcgtgggaacccctcacgagattgccaatgaaacctccgtgaacactcgctacggcgctgagcgcgttattcgctacgcattcgagctggcacagagccgccgcaagaagctcaccctcgtgcacaagaccaacgtcctggttcacggtggtggcctgtggcagcgcaccgtagatgaggttgcaaaggaatacccagaggtagccgtcgattacaaccacatcgatgcagcaaccatctatctggtcactgatccttcccgcttcgatgtgattgttaccgataacctcttcggcgacatcctcaccgatgaggcaggcgcagtctctggcggaattggcctcgcagcatccggcaacatcgatgccacgggcaccaacccttccatgttcgagccagtccacggctctgcaccagatatcgcaggccagggaatcgcagacccaacggcagcaatcctatccgctgcgatgctgctgcgtcacttaggtgatgaagacaacgcagtacgtattgaaacagccatcgcagctgatgtggctggccgagataactctcagccgatttctaccactgaggtgggagaccgcatcgtcaaggcgctgcaaagctaa

SEQ ID NO: 19 - protein, 481 aa, Corynebacterium glutamicum
  leuC
MTSPVENSTSTEKLTLAEKVWRDHVVSKGENGEPDLLYIDLQLLHEVTSPQAFDGLRMTGRKLRHPELHLATEDHNVPTEGIKTGSLLEINDKISRLQVSTLRDNCEEFGVRLHPMGDVRQGIVHTVGPQLGATQPGMTIVCGDSHTSTHGAFGSMAFGIGTSEVEHVMATQTLPLKPFKTMAIEVTGELQPGVSSKDLILAIIAKIGTGGGQGYVLEYRGEAIRKMSMDARMTMCNMSIEAGARAGMIAPDQTTFDYVEGREMAPKGADWDEAVAYWKTLPTDEGATFDKVVEIDGSALTPFITWGTNPGQGLPLGESVPSPEDFTNDNDKAAAEKALQYMDLVPGTPLRDIKIDTVFLGSCTNARIEDLQIAADILKGHKIADGMRMMVVPSSTWIKQEAEALGLDKIFTDAGAEWRTAGCSMCLGMNPDQLKPGERSASTSNRNFEGRQGPGGRTHLVSPAVAAATAIRGTLSSPADI

SEQ ID NO: 20 - DNA, 1446 bp, Corynebacterium glutamicum
  leuC
atgaccagccccgtggagaacagcacctcaactgagaagctgaccctggcagagaaggtgtggcgcgaccatgtcgtgtccaagggagaaaacggcgagcccgacctcctctacatcgacctgcagctgctgcatgaagtgacctcaccacaggcatttgacggcctgcgcatgaccggccgtaaactgcgccacccagaactgcacctggccaccgaagaccacaacgtgccaaccgaaggcatcaagactggctcactgctggaaatcaacgacaagatttcccgcctgcaggtatctactctgcgcgacaactgtgaagaattcggcgtgcgcctgcacccaatgggtgatgtccgacagggcatcgtgcacaccgtcggcccacagctcggcgcaacccagccaggcatgaccattgtgtgcggtgactcccacacctccacccacggtgcttttggctccatggcattcggcatcggtacctcagaggttgagcacgtcatggctactcaaaccctgccactgaagcctttcaagaccatggccattgaagttactggtgaactgcagccaggtgtttcctccaaggacctgattctggcgattatcgccaagatcggcaccggcggcggacagggctacgttctggaataccgcggcgaagcaatccgtaagatgtccatggatgcacgcatgaccatgtgcaacatgtccatcgaagctggcgcacgtgccggcatgatcgccccagaccaaaccaccttcgactacgttgaaggccgcgaaatggcaccaaagggcgccgactgggacgaagcagttgcttactggaagaccctgccaaccgacgaaggcgcaacctttgacaaggtcgtagaaatcgatggctccgcactgaccccattcatcacctggggcaccaacccaggccagggcctgccactgggcgaatccgtaccaagcccagaagacttcaccaacgacaacgacaaggcagcagccgaaaaggcactgcagtacatggacctggtaccaggaaccccactgcgcgacatcaagatcgacaccgtcttcctgggatcctgcaccaacgcccgcatcgaagacctgcagatcgccgctgacatcctcaagggccacaaaatcgccgacggcatgcgcatgatggtcgtgccttcctccacctggatcaagcaagaggcagaagcgctcggactggacaaaatcttcaccgacgctggcgctgaatggcgtaccgcaggctgctccatgtgcctgggcatgaacccagaccaactgaagccaggcgagcgctccgcatccacctccaaccgaaacttcgaaggacgccaaggaccaggaggccgcacccacctggtatccccagcagtcgcagccgccaccgcaatccgcggcaccctgtcctcacctgcagatatctaa

SEQ ID NO: 21 - protein, 197 aa, Corynebacterium glutamicum
  leuD
MEKFTTYTGVGVPLQRSNVDTDQIIPAVYLKRVTRTGFEDGLFSNWRQNDPNFVLNTDTYKNGSVLVAGPDFGTGSSREHAVWALMDYGFRAVFSSRFADIFRGNSGKAGMLTGIMEQSDIELLWKLMEQTPGLELTVNLEKQIVTAGDVVISFEVDPYIRWRLMEGLDDAGLTLRKLDEIEDYEAKRPAFKPRTNA

SEQ ID NO: 22 - DNA, 594 bp, Corynebacterium glutamicum
  leuD
atggaaaaatttaccacctacaccggcgttggcgttccactgcagcgatccaacgtggacaccgaccagatcatcccagccgtctacctcaagcgcgtcacccggaccggcttcgaagacggactgttttccaactggcgccaaaacgaccccaactttgtcctcaacaccgacacctacaagaacggctccgttctcgtagcaggccctgactttggcaccggctcctcccgcgagcacgccgtctgggcactcatggactacggcttccgcgctgtcttctcctcacgattcgccgacatcttccgcggcaactccggaaaagcgggcatgctcaccggcatcatggaacagtccgacatcgaacttctgtggaagctcatggaacaaaccccaggcctcgaactgaccgtgaacctggaaaagcagatcgtcaccgcaggcgacgtagtgatcagcttcgaagttgacccctacatccgctggcgtttgatggaaggcctcgacgacgctggcctgaccctgcgcaagctcgatgaaattgaagactacgaggctaagcgccctgcgtttaagccacgcactaacgcttaa

SEQ ID NO: 23 - protein, 436 aa, Corynebacterium glutamicum
  ilvA
MSETYVSEKSPGVMASGAELIRAADIQTAQARISSVIAPTPLQYCPRLSEETGAEIYLKREDLQDVRSYKIRGALNSGAQLTQEQRDAGIVAASAGNHAQGVAYVCKSLGVQGRIYVPVQTPKQKRDRIMVHGGEFVSLVVTGNNFDEASAAAHEDAERTGATLIEPFDARNTVIGQGTVAAEILSQLTSMGKSADHVMVPVGGGGLLAGVVSYMADMAPRTAIVGIEPAGAASMQAALHNGGPITLETVDPFVDGAAVKRVGDLNYTIVEKNQGRVHMMSATEGAVCTEMLDLYQNEGIIAEPAGALSIAGLKEMSFAPGSVVVCIISGGNNDVLRYAEIAERSLVHRGLKHYFLVNFPQKPGQLRHFLEDILGPDDDITLFEYLKRNNRETGTALVGIHLSEASGLDSLLERMEESAIDSRRLEPGTPEYEYLT

SEQ ID NO: 24 - DNA, 1311 bp, Corynebacterium glutamicum
  aceE
atgagtgaaacatacgtgtctgagaaaagtccaggagtgatggctagcggagcggagctgattcgtgccgccgacattcaaacggcgcaggcacgaatttcctccgtcattgcaccaactccattgcagtattgccctcgtctttctgaggaaaccggagcggaaatctaccttaagcgtgaggatctgcaggatgttcgttcctacaagatccgcggtgcgctgaactctggagcgcagctcacccaagagcagcgcgatgcaggtatcgttgccgcatctgcaggtaaccatgcccagggcgtggcctatgtgtgcaagtccttgggcgttcagggacgcatctatgttcctgtgcagactccaaagcaaaagcgtgaccgcatcatggttcacggcggagagtttgtctccttggtggtcactggcaataacttcgacgaagcatcggctgcagcgcatgaagatgcagagcgcaccggcgcaacgctgatcgagcctttcgatgctcgcaacaccgtcatcggtcagggcaccgtggctgctgagatcttgtcgcagctgacttccatgggcaagagtgcagatcacgtgatggttccagtcggcggtggcggacttcttgcaggtgtggtcagctacatggctgatatggcacctcgcactgcgatcgttggtatcgaaccagcgggagcagcatccatgcaggctgcattgcacaatggtggaccaatcactttggagactgttgatccctttgtggacggcgcagcagtcaaacgtgtcggagatctcaactacaccatcgtggagaagaaccagggtcgcgtgcacatgatgagcgcgaccgagggcgctgtgtgtactgagatgctcgatctttaccaaaacgaaggcatcatcgcggagcctgctggcgcgctgtctatcgctgggttgaaggaaatgtcctttgcacctggttctgtcgtggtgtgcatcatctctggtggcaacaacgatgtgctgcgttatgcggaaatcgctgagcgctccttggtgcaccgcggtttgaagcactacttcttggtgaacttcccgcaaaagcctggtcagttgcgtcacttcctggaagatatcctgggaccggatgatgacatcacgctgtttgagtacctcaagcgcaacaaccgtgagaccggtactgcgttggtgggtattcacttgagtgaagcatcaggattggattctttgctggaacgtatggaggaatcggcaattgattcccgtcgcctcgagccgggcacccctgagtacgaatacttgacctaa

SEQ ID NO: 25 - protein, 922 aa, Corynebacterium glutamicum
  aceE
MADQAKLGGKPSDDSNFAMIRDGVASYLNDSDPEETNEWMDSLDGLLQESSPERARYLMLRLLERASAKRVSLPPMTSTDYVNTIPTSMEPEFPGDEEMEKRYRRWIRWNAAIMVHRAQRPGIGVGGHISTYAGAAPLYEVGFNHFFRGKDHPGGGDQIFFQGHASPGMYARAFMEGRLSEDDLDGFRQEVSREQGGIPSYPHPHGMKDFWEFPTVSMGLGPMDAIYQARFNRYLENRGIKDTSDQHVWAFLGDGEMDEPESRGLIQQAALNNLDNLTFVVNCNLQRLDGPVRGNTKIIQELESFFRGAGWSVIKVVWGREWDELLEKDQDGALVEIMNNTSDGDYQTFKANDGAYVREHFFGRDPRTAKLVENMTDEEIWKLPRGGHDYRKVYAAYKRALETKDRPTVILAHTIKGYGLGHNFEGRNATHQMKKLTLDDLKLFRDKQGIPITDEQLEKDPYLPPYYHPGEDAPEIKYMKERRAALGGYLPERRENYDPIQVPPLDKLRSVRKGSGKQQIATTMATVRTFKELMRDKGLADRLVPIIPDEARTFGLDSWFPTLKIYNPHGQNYVPVDHDLMLSYREAPEGQILHEGINEAGSVASFIAAGTSYATHGKAMIPLYIFYSMFGFQRTGDSIWAAADQMARGFLLGATAGRTTLTGEGLQHMDGHSPVLASTNEGVETYDPSFAYEIAHLVHRGIDRMYGPGKGEDVIYYITIYNEPTPQPAEPEGLDVEGLHKGIYLYSRGEGTGHEANILASGVGMQWALKAASILEADYGVRANIYSATSWVNLARDGAARNKAQLRNPGADAGEAFVTTQLKQTSGPYVAVSDFSTDLPNQIREWVPGDYTVLGADGFGFSDTRPAARRFFNIDAESIVVAVLNSLAREGKIDVSVAAQAAEKFKLDDPTSVSVDPNAPEE

SEQ ID NO: 26 - DNA, 2769 bp, Corynebacterium glutamicum
atggccgatcaagcaaaacttggtggcaagccctcggatgactctaacttcgcgatgatccgcgatggcgtggcatcttatttgaacgactcagatccggaggagaccaacgagtggatggattcactcgacggattactccaggagtcttctccagaacgtgctcgttacctcatgcttcgtttgcttgagcgtgcatctgcaaagcgcgtatctcttcccccaatgacgtcaaccgactacgtcaacaccattccaacctctatggaacctgaattcccaggcgatgaggaaatggagaagcgttaccgtcgttggattcgctggaacgcagccatcatggttcaccgcgctcagcgaccaggcatcggcgtcggcggacacatttccacttacgcaggcgcagcccctctgtacgaagttggcttcaaccacttcttccgcggcaaggatcacccaggcggcggcgaccagatcttcttccagggccacgcatcaccaggtatgtacgcacgtgcattcatggagggtcgcctttctgaagacgatctcgatggcttccgtcaggaagtttcccgtgagcagggtggcattccgtcctaccctcacccacacggtatgaaggacttctgggagttcccaactgtgtccatgggtcttggcccaatggatgccatttaccaggcacgtttcaaccgctacctcgaaaaccgtggcatcaaggacacctctgaccagcacgtctgggccttccttggcgacggcgaaatggacgagccagaatcacgtggtctcatccagcaggctgcactgaacaacctggacaacctgaccttcgtggttaactgcaacctgcagcgtctcgacggacctgtccgcggtaacaccaagatcatccaggaactcgagtccttcttccgtggcgcaggctggtctgtgatcaaggttgtttggggtcgcgagtgggatgaacttctggagaaggaccaggatggtgcacttgttgagatcatgaacaacacctccgatggtgactaccagaccttcaaggctaacgacggcgcatatgttcgtgagcacttcttcggacgtgacccacgcaccgcaaagctcgttgagaacatgaccgacgaagaaatctggaagcttccacgtggcggccacgattaccgcaaggtttacgcagcctacaagcgagctcttgagaccaaggatcgcccaaccgtcatccttgctcacaccattaagggctacggactcggccacaacttcgaaggccgtaacgcaacccaccagatgaagaagctgacgcttgatgatctgaagttgttccgcgacaagcagggcatcccaatcaccgatgagcagctggagaaggatccttaccttcctccttactaccacccaggtgaagacgctcctgaaatcaagtacatgaaggaacgtcgcgcagcgctcggtggctacctgccagagcgtcgtgagaactacgatccaattcaggttccaccactggataagcttcgctctgtccgtaagggctccggcaagcagcagatcgctaccaccatggcgactgttcgtaccttcaaggaactgatgcgcgataagggcttggctgatcgccttgtcccaatcattcctgatgaggcacgtaccttcggtcttgactcttggttcccaaccttgaagatctacaacccgcacggtcagaactacgtgcctgttgaccacgacctgatgctctcctaccgtgaggcacctgaaggacagatcctgcacgaaggcatcaacgaggctggttccgtggcatcgttcatcgctgcgggtacctcctacgccacccacggcaaggccatgattccgctgtacatcttctactcgatgttcggattccagcgcaccggtgactccatctgggcagcagccgatcagatggcacgtggcttcctcttgggcgctaccgcaggtcgcaccaccctgaccggtgaaggcctccagcacatggatggacactcccctgtcttggcttccaccaacgagggtgtcgagacctacgacccatcctttgcgtacgagatcgcacacctggttcaccgtggcatcgaccgcatgtacggcccaggcaagggtgaagatgttatctactacatcaccatctacaacgagccaaccccacagccagctgagccagaaggactggacgtagaaggcctgcacaagggcatctacctctactcccgcggtgaaggcaccggccatgaggcaaacatcttggcttccggtgttggtatgcagtgggctctcaaggctgcatccatccttgaggctgactacggagttcgtgccaacatttactccgctacttcttgggttaacttggctcgcgatggcgctgctcgtaacaaggcacagctgcgcaacccaggtgcagatgctggcgaggcattcgtaaccacccagctgaagcagacctccggcccatacgttgcagtgtctgacttctccactgatctgccaaaccagatccgtgaatgggtcccaggcgactacaccgttctcggtgcagatggcttcggtttctctgatacccgcccagctgctcgtcgcttcttcaacatcgacgctgagtccattgttgttgcagtgctgaactccctggcacgcgaaggcaagatcgacgtctccgttgctgctcaggctgctgagaagttcaagttggatgatcctacgagtgtttccgtagatccaaacgctcctgaggaataa

SEQ ID NO: 27 - DNA, 28 bp, Artificial Sequence
  Description of Artificial Sequence Synthetic Pcj7-5-KpnI oligonucleotide
gatggtaccaccccagaaacatcccagc

SEQ ID NO: 28 - DNA, 30 bp, Artificial Sequence
  Description of Artificial Sequence Synthetic Pcj7-3 NdeI oligonucleotide
cgatcatatggagtgtttcctttcgttggg

SEQ ID NO: 29 - DNA, 21 bp, Artificial Sequence
  Description of Artificial Sequence Synthetic 117-F oligonucleotide
ccacagccgacaggatggtga

SEQ ID NO: 30 - DNA, 21 bp, Artificial Sequence
  Description of Artificial Sequence Synthetic 117-R oligonucleotide
ctcagggtgtagcggttcggt

SEQ ID NO: 31 - DNA, 34 bp, Artificial Sequence
  Description of Artificial Sequence Synthetic leuB-5-cimA primer
ttgttgaattaatctagaggtgacaccccagtgg

SEQ ID NO: 32 - DNA, 34 bp, Artificial Sequence
  Description of Artificial Sequence Synthetic leuB-3-leuC primer
tcgcagctgccaccgatatttagctttgcagcgc

SEQ ID NO: 33 - DNA, 34 bp, Artificial Sequence
  Description of Artificial Sequence Synthetic leuC-5-leuB oligonucleotide
gcgctgcaaagctaaatatcggtggcagctgcga

SEQ ID NO: 34 - DNA, 34 bp, Artificial Sequence
  Description of Artificial Sequence Synthetic leuD-3-XbaI oligonucleotide
tggcggccgctctagagctttcgctatcagactg

SEQ ID NO: 35 - DNA, 25 bp, Artificial Sequence
  Description of Artificial Sequence Synthetic cimA-5-NdeI oligonucleotide
gcatcatatgatggtaaggatattc

SEQ ID NO: 36 - DNA, 27 bp, Artificial Sequence
  Description of Artificial Sequence Synthetic cimA-3-XbaI oligonucleotide
cgatctagattaattcaacaacatgtt

SEQ ID NO: 37 - DNA, 27 bp, Artificial Sequence
  Description of Artificial Sequence Synthetic ilvA-5-XbaI oligonucleotide
gcatctagagaacacggacaatgccac

SEQ ID NO: 38 - DNA, 32 bp, Artificial Sequence
  Description of Artificial Sequence Synthetic ilvA-Del-R oligonucleotide
caaacagcgtgatgtcctgagtgagctgcgct

SEQ ID NO: 39 - DNA, 32 bp, Artificial Sequence
  Description of Artificial Sequence Synthetic ilvA-Del-F oligonucleotide
agcgcagctcactcaggacatcacgctgtttg

SEQ ID NO: 40 - DNA, 29 bp, Artificial Sequence
  Description of Artificial Sequence Synthetic ilvA-3-XbaI oligonucleotide
gcatctagaacctgcggcacaccttgggc

SEQ ID NO: 41 - DNA, 19 bp, Artificial Sequence
  Description of Artificial Sequence Synthetic Topo-F oligonucleotide
gcagtgagcgcaacgcaat

SEQ ID NO: 42 - DNA, 19 bp, Artificial Sequence
  Description of Artificial Sequence Synthetic Topo-R oligonucleotide
cgttgtaaaacgacggcca

SEQ ID NO: 43 - DNA, 19 bp, Artificial Sequence
  Description of Artificial Sequence Synthetic CFilvA oligonucleotide
gatctgtgatgaggtgatg

SEQ ID NO: 44 - DNA, 19 bp, Artificial Sequence
  Description of Artificial Sequence Synthetic CRilvA oligonucleotide
cgtccttccatgactctca

SEQ ID NO: 45 - DNA, 26 bp, Artificial Sequence
  Description of Artificial Sequence Synthetic lysC-5-KpnI oligonucleotide
tgaggtacccatgcgattgttaatgc

SEQ ID NO: 46 - DNA, 28 bp, Artificial Sequence
  Description of Artificial Sequence Synthetic asd-3-SfoI oligonucleotide
ctaggcgccagtgtagcactcaagcgga

SEQ ID NO: 47 - DNA, 27 bp, Artificial Sequence
  Description of Artificial Sequence Synthetic hom-5-BamHI oligonucleotide
ctaggatccctcaccattctcaatggt

SEQ ID NO: 48 - DNA, 25 bp, Artificial Sequence
  Description of Artificial Sequence Synthetic thrB-3-BamHI oligonucleotide
ctaggatccgccttccttgttgggc

SEQ ID NO: 49 - DNA, 27 bp, Artificial Sequence
  Description of Artificial Sequence Synthetic thrC-5-SpeI oligonucleotide
tgcactagtgagaacatacaggttcca

SEQ ID NO: 50 - DNA, 32 bp, Artificial Sequence
  Description of Artificial Sequence Synthetic thrC-3-ilvA oligonucleotide
ggcattgtccgtgttcttacttcacggaagtg

SEQ ID NO: 51 - DNA, 32 bp, Artificial Sequence
  Description of Artificial Sequence Synthetic ilvA-5-thrC oligonucleotide
cacttccgtgaagtaagaacacggacaatgcc

SEQ ID NO: 52 - DNA, 29 bp, Artificial Sequence
  Description of Artificial Sequence Synthetic ilvA-3-SpeI oligonucleotide
tgcactagtacctgcggcacaccttgggc